(19) United States
(12) Patent Application Publication
(10) Pub. No.: US 2010/0114184 A1
(43) Pub. Date: May 6, 2010

(54) FLEXIBLE TOOLS FOR PREPARING BONY CANALS

(75) Inventors: Leonid Degtyar, Azor (IL); Tal Reich, Rishon Le Zion (IL)

Correspondence Address: RATNERPRESTIA, P.O. BOX 980, VALLEY FORGE, PA, 19482, US

(73) Assignee: BRAINSGATE LTD., Caesarea (IL)

(21) Appl. No.: 12/575,165

(22) Filed: Oct. 7, 2009

Related U.S. Application Data
(60) Provisional application No. 61/195,556, filed on Oct. 7, 2008.

(51) Int. Cl. A61B 17/56

(52) U.S. Cl. 606/86 R

(57) ABSTRACT

Apparatus is provided that includes a surgical tool, which includes a proximal shaft, a distal rod, and a target sight. A proximal end of the distal rod is coupled to a distal end of the proximal shaft such that the distal rod articulates with the proximal shaft. The target sight includes an aiming element, which is coupled to the distal rod and extends toward the proximal shaft, and which is indicative of an alignment of the distal rod with respect to the proximal shaft. Other embodiments are also described.

### CROSS-REFERENCE TO RELATED APPLICATIONS

The present application claims the benefit of U.S. Provisional Application 61/195,556, filed Oct. 7, 2008, which is assigned to the assignee of the present application and is incorporated herein by reference.

### FIELD OF THE INVENTION

The present invention relates generally to medical procedures and tools. More specifically, the invention relates to medical procedures and tools for preparing a bony canal for implantation therethrough of a medical device.

### BACKGROUND OF THE INVENTION

The sphenopalatine ganglion (SPG) is a neuronal center located in the brain behind the nose. It consists of parasympathetic neurons innervating the middle cerebral and anterior cerebral lumens, the facial skin blood vessels, and the lacrimal glands. Activation of this ganglion is believed to cause vasodilation of these vessels. A second effect of such stimulation is the opening of pores in the vessel walls, causing plasma protein extravasation (PPE). This effect allows better transport of molecules from within these blood vessels to surrounding tissue.

PCT Publication WO 01/85094 to Shalev et al., and U.S. Pat. No. 7,120,489 in the national stage thereof, which are incorporated herein by reference, describe methods and apparatus for stimulating the SPG to modify properties of the blood brain barrier and cerebral blood flow for the treatment of medical conditions. Treatment is accomplished directly via stimulation of the sphenopalatine ganglion and/or indirectly by the facilitation of drug transport across the blood brain barrier via stimulation of the sphenopalatine ganglion.

U.S. Pat. No. 6,526,318 to Ansarinia and related PCT Patent Publication WO 01/97905 to Ansarinia, which are incorporated herein by reference, describe a method for treating a patient by placing at least one electrode on or proximate to at least one of the patient's sphenopalatine ganglia, sphenopalatine nerves, or vidian nerves, and activating the electrode to apply an electrical signal and/or a medical solution to at least one of those ganglia or nerves. The '318 patent and '905 publication also describe surgical techniques for implanting the electrode via a coronoid notch of the patient.

U.S. Pat. No. 6,405,079 to Ansarinia, which is incorporated herein by reference, describes methods for treating medical conditions by implanting one or more electrodes in regions of the sinus and applying electrical stimulation and/or medical solutions to the implantation site. The '079 patent also describes surgical techniques for implanting the electrode.

PCT Publication WO 04/043218 to Gross et al., and US Patent Application Publication 2006/0195169 in the national stage thereof, which are incorporated herein by reference, describe apparatus for treating a subject, comprising (a) a stimulation device, adapted to be implanted in a vicinity of a site selected from the list consisting of: a SPG of the subject and a neural tract originating in or leading to the SPG; and (b) a connecting element, coupled to the stimulation device, and adapted to be passed through at least a portion of a greater palatine canal of the subject. Also provided is a method for implanting a treatment stimulation device in a vicinity of a site of a subject, the method comprising passing the device through a greater palatine foramen of the subject, and bringing the device into contact with the vicinity of the site, the site selected from the list consisting of: a SPG of the subject and a neural tract originating in or leading to the SPG.

U.S. Pat. No. 7,117,033 to Shalev et al., which is incorporated herein by reference, describes a method for treating a subject, comprising positioning at least one electrode at least one site of the subject for less than about 3 hours, applying an electrical current to the site of the subject, and configuring the current to increase cerebral blood flow (CBF) of the subject, so as to treat a condition of the subject. The site is selected from the list consisting of: a sphenopalatine ganglion (SPG), a greater palatine nerve, a lesser palatine nerve, a sphenopalatine nerve, a communicating branch between a maxillary nerve and an SPG, an otic ganglion, an afferent fiber going into the otic ganglion, an efferent fiber going out of the otic ganglion, an infraorbital nerve, a vidian nerve, a greater superficial petrosal nerve, and a lesser deep petrosal nerve. Also described is an apparatus comprising an elongated support element having a length of between about 1.8 cm and about 4 cm, and having a proximal end and a distal end; one or more electrodes fixed to the support element in a vicinity of the distal end thereof; a receiver, fixed to the support element in a vicinity of the proximal end thereof; and a control unit, adapted to be coupled to the receiver, and adapted to drive the electrodes to apply an electrical current to tissue of the subject, and configure the current to have a pulse frequency of between about 10 Hz and about 50 Hz, an amplitude of between about 0.2 V and about 10 V, a pulse width of between about 50 microseconds and about 5 milliseconds, and, in alternation, on periods of between about 1 second and about 2 minutes, and off periods of between about 1 second and about 2 minutes.

The following patents and patent application publications, all of which are incorporated herein by reference, may be of interest: U.S. Pat. No. 6,853,858 to Shalev, U.S. Pat. No. 7,146,209, US Patent Application Publication 2006/0287677 to Shalev et al., US Patent Application Publication 2007/0083245 to Lamensdorf et al., PCT Publication WO 03/090599, PCT Publication WO 03/105658, PCT Publication WO 04/01092, PCT Publication WO 04/044947, PCT Publication WO 04/045242, PCT Publication WO 04/043217, PCT Publication WO 04/043334, PCT Publication WO 05/030025, and PCT Publication WO 05/030118.

U.S. Pat. No. 5,766,605 to Sanders et al. describes a method for the control of autonomic nerve function in a mammal comprising administering a therapeutically effective amount of botulinum toxin to the mammal. Preferred embodiments include administering the toxin to control the function of an autonomic nerve which contributes to at least one symptom of rhinorrhea, otitis media, excessive salivation, asthma, COPD, excessive stomach acid secretion, spastic colitis or excessive sweating.

U.S. Pat. No. 5,697,377 to Wittkampf describes techniques for catheter location mapping, and related procedures. Three substantially orthogonal alternating signals are applied through the patient, directed substantially toward the area of interest to be mapped, such as patient's heart. The currents are preferably constant current pulses, of a frequency and magnitude to avoid disruption with ECG recordings. A catheter is equipped with at least a measuring electrode, which for cardiac procedures is positioned at various locations either against the patient's heart wall, or within a coronary vein or artery. A voltage is sensed between the catheter tip and a reference electrode, preferably a surface electrode on the patient, which voltage signal has components corresponding to the three orthogonal applied current signals. Three processing channels are used to separate out the three components as x, y and z signals, from which calculations are made for determination of the three-dimensional location of the catheter tip within the body. An easy calibration procedure, which can be performed separately or during the mapping, is used to calibrate the system and provide the correlations between respective x, y and z sense signals and dimensional locations. The procedure is particularly applicable for catheter mapping prior to ablation, and for repositioning the catheter tip at precise locations for the desired ablations. The procedure is also applicable for other techniques where position must be remembered and re-found with accuracy, such as in mapping coronary stenosis and/or placing stents. Although the invention provides the greatest benefit in 3-dimensional applications, it is also useful for one and two dimensional applications.

US Patent Application Publication 2006/0154199 to Maxwell et al. describes a wireless dental apex locator for use in determining the location of the apex of a patient's root. The locator includes an electronic module having a battery power source, an impedance analyzer circuit and a radio frequency transmitter; a grounding module having a clip for grounding the patient; a probe module having an endodontic probe; and an associated, but not physically connected, display unit having a receiver for receiving radio frequency signals from the transmitter. The display unit has an electronic circuit that conditions the signals and interprets the signal for display on a graphic display.

The following references may be of interest:
U.S. Pat. No. 4,867,164 to Zabara
U.S. Pat. No. 5,314,495 to Kovacs
U.S. Pat. No. 5,453,575 to O'Donnell et al.
U.S. Pat. No. 5,792,100 to Shantha
U.S. Pat. No. 6,343,226 to Sunde et al.
U.S. Pat. No. 6,432,986 to Levin
U.S. Pat. No. 6,606,521 to Paspa et al.
U.S. Pat. No. 6,735,475 to Whitehurst et al.
U.S. Pat. No. 6,782,292 to Whitehurst
U.S. Pat. No. 6,788,975 to Whitehurst et al.
U.S. Pat. No. 6,860,264 to Christopher
US Patent Application Publication 2002/0077687 to Ahn
US Patent Application Publication 2003/0050527 to Fox et al.
US Patent Application Publication 2004/0138097 to Guyuron
US Patent Application Publication 2005/0065427 to Magill et al.
US Patent Application Publication 2005/0113877 to Spinelli et al.
US Patent Application Publication 2006/0293723 to Whitehurst et al.
Branston N M, “The physiology of the cerebrovascular parasympathetic innervation,” British Journal of Neurosurgery 9:319-329 (1995)
Nissen S E et al., “Effect of Very High-Intensity Statin Therapy on Regression of Coronary Atherosclerosis The ASTEROID Trial,” JAMA 295(13):1556-1565 (2006)
Seylaz J et al., “Effect of stimulation of the sphenopalatine ganglion on cortical blood flow in the rat,” J Cereb Blood Flow Metab 8(6):875-8 (1988)
Suzuki N et al., “Selective electrical stimulation of postganglionic cerebrovascular parasympathetic nerve fibers originating from the sphenopalatine ganglion enhances cortical blood flow in the rat,” Journal of Cerebral Blood Flow and Metabolism, 10, 383-391 (1990)
Suzuki N et al., “Effect on cortical blood flow of electrical stimulation of trigeminal cerebrovascular nerve fibres in the rat,” Acta Physiol. Scand., 138, 307-315 (1990)
Toda N et al., “Cerebral vasodilation induced by stimulation of the pterygopalatine ganglion and greater petrosal nerve in anesthetized monkeys,” Neuroscience 96(2):393-398 (2000)
Walters B B et al., “Cerebrovascular projections from the sphenopalatine and otic ganglia to the middle cerebral artery of the cat,” Stroke 17:488-494 (1986)
Wong J D et al., “Maxillary nerve block anaesthesia via the greater palatine canal: A modified technique and case reports,” Australian Dental Journal 36(1):15-21 (1991)

### SUMMARY OF THE INVENTION

In some embodiments of the present invention, surgical tools and methods are provided for preparing a greater palatine canal to allow placement of an elongated neural stimulator in and/or through the canal. The stimulator is typically used to apply stimulation to the sphenopalatine ganglion (SPG), which is located at the distal end of the canal. The tools are configured to open a passage through the canal while minimizing the application of forces in directions perpendicular to the longitudinal axes of the tools. Such perpendicular forces could damage fragile anterior, left, and right walls of the canal. The tools typically open the passage through soft tissue within the canal, and/or between anatomical features of the canal, such as between soft tissue and the osseous wall of the canal. In some cases, the tools dilate the canal. The tools include a forward tool and an abrading tool.

In some embodiments of the present invention, a forward tool is configured to apply a forward longitudinal force in order to open the passage through the canal, substantially without applying forces to the walls of the canal in directions perpendicular to the longitudinal axis of the tool. The forward tool comprises a distal rod, a proximal shaft, and a resisting element. The distal rod is coupled to the proximal shaft such that the distal rod articulates with the proximal shaft with greater than one degree of freedom, such as in all directions (i.e., 360 degrees), while the resisting element is arranged to resist articulation of the distal rod with the proximal shaft. The distal rod is shaped so as to define a blunt distal tip that opens the passage the canal as the tip is advanced distally through the canal and applies the forward longitudinal force. The articulation minimizes the application of forces to the walls of the canal in directions perpendicular to the longitudinal axis of the distal rod, such as if the proximal shaft should become slightly misaligned with the direction of the canal. Typically, the resisting element couples the proximal shaft to the distal rod. The resisting element typically comprises an elastic element.

In some embodiments of the present invention, an abrading tool is configured to both apply a forward longitudinal force and abrade in a posterior direction, substantially without applying forces to the left and right walls of the canal perpendicular to the abrasion direction. The abrading tool comprises a distal rod, a proximal shaft, and a resisting element. The distal rod is coupled to the proximal shaft such that the distal rod articulates with the proximal shaft with exactly one degree of freedom. As a result, the distal rod articulates in the left and right directions, and is prevented from articulating in the posterior and anterior directions perpendicular to these two directions. The resisting element is arranged to resist articulation of the distal rod with the proximal shaft. A portion of the abrading tool facing in the posterior direction is shaped so as to define an abrading surface. This arrangement provides rigidity in the posterior direction for abrading, while providing flexibility in the left and right directions, thereby minimizing the application of forces to the left and right walls of the canal in respective directions perpendicular to the axis of the distal rod. Typically, the resisting element couples the proximal shaft to the distal rod. The resisting element typically comprises an elastic element.

The forward and abrading tools generally provide the surgeon performing the implantation procedure with greater flexibility regarding the angle of the approach to the canal. The increased freedom of motion provided by the articulation of the tools allows the surgeon to maneuver more easily within the mouth of the patient.

In some embodiments of the present invention, the proximal shaft is shaped so as to define a bend having an angle of between 90 and 175 degrees, such as 160 degrees. The bend is ergonomically helpful to the surgeon performing the implantation procedure. For some applications, the distal rod is shaped so as to define a hollow area in a vicinity of the proximal end thereof. At least a portion of the resisting element is positioned within the hollow area. This hollow area helps accommodate the resisting element within the portion of the tool distal to the bend, while, for some applications, at the same time limiting the articulation of the distal element with the proximal shaft. For some applications, an external wall of the hollow area is shaped so as to define at least one opening therethrough, which may facilitate cleaning of the hollow area.

In some embodiments of the present invention, the forward tool and/or abrading tool comprises a target sight. The sight helps the surgeon aim the tool as the tool is advanced through the canal. The sight comprises an aiming element that is coupled to the distal rod, and extends in a generally proximal direction toward the proximal shaft, past an articulation site at which the distal rod articulates with the proximal shaft. The aiming element, and a proximal guide tip thereof, move with respect to the proximal shaft as the distal rod articulates with the proximal shaft. The aiming element thus is indicative of the alignment of the distal rod with respect to the proximal shaft.

For some applications, the sight further comprises an aiming ring, which is coupled to the proximal shaft. The aiming ring and the aiming element are arranged such that the proximal guide tip of the aiming element can easily be observed with respect to the aiming ring. Typically, the proximal guide tip is approximately centered with respect to the aiming ring when the distal rod is aligned with the proximal shaft. As the distal rod articulates in with respect to the proximal shaft, the proximal guide tip moves in a generally opposite direction with respect to the aiming ring.

In some embodiments of the present invention, a surgeon uses the above-mentioned tools to perform a surgical procedure to dilate and shape the greater palatine canal to a desired geometry. A surgical punch is used to puncture the mucosa covering a greater palatine foramen, providing access to the greater palatine canal and foramen. The forward tool is advanced into greater palatine canal, and is used to apply a forward longitudinal force in order to open a passage through the canal.

A series of abrading tools, having successively greater diameters, is typically used to widen the path through the canal created using the forward tool. First, the narrowest abrading tool of the series is introduced through the path created by the forward tool, keeping tight contact between the instrument and a posterior wall of the greater palatine canal. This dilating step of the surgical procedure is repeated using abrading tools having successively greater diameters, until the greater palatine canal is widened, typically, to about 2 mm. For some applications, the series of abrading tools includes exactly three tools, having diameters of 1.5 mm, 1.8 mm, and 2.0 mm, respectively.

In embodiments in which the tool comprises the sight, during the implantation procedure, as the surgeon advances the distal rod through the canal, he or she monitors the position of the proximal guide tip with respect to the aiming ring. If the proximal guide tip becomes deflected from center in a given direction, the surgeon moves the proximal shaft in this given direction, thereby bringing the guide tip back to center. As a result, the proximal shaft becomes realigned with the distal rod and the direction of the canal, thereby reducing the risk of the distal rod penetrating the wall of the canal.

A neural stimulator is typically implanted in the greater palatine canal after the passage has been opened through the canal. Once implanted, the stimulator typically delivers energy to the SPG or another parasympathetic site (such as those described in the patent applications incorporated hereinbelow by reference) in order to activate the SPG or other site, to control and/or modify SPG-related behavior, e.g., in order to induce changes in cerebral blood flow and/or to modulate permeability of the blood-brain-barrier (BBB).

The stimulation may be used in many medical applications, such as, by way of illustration and not limitation:
the treatment or prevention of cerebrovascular disorders such as stroke or other adverse brain events, such as described in U.S. application Ser. No. 11/465,381;
the enhancement of neurogenesis or brain metabolic activity, such as described in U.S. application Ser. No. 12/197,614;
the treatment or prevention of vascular dementia (VaD), such as described in U.S. application Ser. No. 11/874,529;
the treatment or prevention of Alzheimer's disease, such as described in U.S. application Ser. No. 10/518,322 and/or U.S. application Ser. No. 11/874,529;
the facilitation of drug transport across the BBB, such as described in U.S. Pat. No. 7,120,489.

Alternatively, the stimulator is configured to inhibit the SPG or other site, such as in order to treat migraine headaches.

Although the surgical tools and methods described herein have been described as being used for opening a passage through the greater palatine canal, these tools and methods may also be used for opening passages through other bony canals, such as the incisive canal.

In some embodiments of the present invention, a probe system is provided for ascertaining whether a probe has been properly positioned within a bony canal of a subject, such as a greater palatine canal, rather than in adjacent tissue outside of the canal. The probe comprises a shaft and a position assessment element coupled thereto. The shaft with the position assessment element is introduced into a body of the subject in a vicinity of the bony canal, and the probe system ascertains whether the position assessment element is within or outside the bony canal. For example, the position assessment element may be outside of the bony canal if all or a portion of the shaft inadvertently never entered the proximal end of the canal, or accidentally punctured through the wall of the canal while being advanced. Typically, the distal end of the shaft is advanced up to about 20 mm through the canal, such that the shaft does not exit the distal end of the canal. Typically, once proper positioning has been ascertained, the probe is removed from the canal, and a neural stimulator is introduced into the bony canal in the same position the probe previously occupied.

In some embodiments of the present invention, the probe comprises an ultrasound probe, which comprises a shaft and one or more ultrasound transducers fixed to the shaft, typically within 10 mm of a distal end thereof. The probe system comprises an external position assessment unit that processes signals produced by the ultrasound transducer to differentiate between bone and softer tissue. If the probe is properly positioned with the bony canal, the transducer will detect the bony wall of the canal. On the other hand, if the probe penetrates the wall of the canal into soft tissue surrounding the canal, the transducer will detect the softer tissue surrounding the canal.

In some embodiments of the present invention, the probe is configured to emit light, typically from within 10 mm of a distal end of the shaft. If the shaft is properly positioned in the bony canal, no or very little of the light is detectable from within the mouth or nose, or on an external surface of the face. On the other hand, if the shaft penetrates the wall of the canal into soft tissue surrounding the canal, the light is detectable from within the mouth or nose, or on the external surface of the face. For some applications, a light sensor is provided for placement in the mouth or nose, or on the external surface of the face to aid with detection of the light.

In some embodiments of the present invention, the probe comprises a balloon, typically positioned within 10 mm of a distal end of the shaft. The probe system comprises a pressure-regulated source of fluid, which is in fluid communication with the balloon via the shaft of the probe. After the probe is inserted into the body canal, the fluid source inflates the balloon with a volume of the fluid, while measuring the applied pressure. The probe system analyzes (a) the volume and/or the rate of change of the volume and (b) the measured pressure of the provided fluid to ascertain whether the probe is properly positioned in the body canal. If the probe is properly positioned in the bony canal, the ratio of pressure to volume is relatively high. On the other hand, if the probe penetrates the wall of the canal into soft tissue surrounding the canal, the ratio of pressure to volume is relatively low.

In some embodiments of the present invention, the shaft is shaped so as to define a channel that extends to an opening, typically within 10 mm of a distal end of the shaft. The probe system comprises a pressure-regulated source of fluid, which is in fluid communication with the channel. After the shaft is inserted into the body canal, the fluid source injects a volume of fluid into the bony canal via the channel and the opening, while measuring the applied pressure. The probe system analyzes (a) the volume and/or the rate of change of the volume and (b) the measured pressure of provided fluid to ascertain whether the shaft is properly positioned in the body canal. If the shaft is properly positioned in the bony canal, the ratio of pressure to volume is relatively high. On the other hand, if the shaft penetrates the wall of the canal into soft tissue surrounding the canal, the ratio of pressure to volume is relatively low.

These techniques enable quick and accurate detection of the location of the bony canal. These techniques are thus generally useful for implantation of neural stimulators in the bony canal, and may be particularly useful for urgent implantation, such as during emergency treatment during the first several hours after stroke.

In some embodiments of the present invention, a method is provided for placing at least one electrode at a desired implantation location in a vicinity of a bony canal of a subject, such as a greater palatine canal. The electrode is advanced at least partially through the bony canal, while electrically activated. The anatomical and electrophysiological conditions surrounding the electrode change as the electrode passes through the bony canal and out of a distal end of the canal. In order to detect these changing conditions, an electrical parameter is sensed at one or more sensing sites on an external surface of the subject's body. Responsively to a change in the electrical parameter, passage of the electrode out of the distal end of the canal is detected, indicating that the electrode (a) entered the proximal end of the canal, and (b) did not accidentally puncture through the wall of the canal. The desired implantation location is ascertained responsively to detection of the passage. For some applications, the electrode is implanted at the implantation location. For other applications, the electrode is a temporary electrode, which is withdrawn from the canal, and an implantable electrode is subsequently implanted at the ascertained implantation location.

In some embodiments of the present invention, the bony canal is the greater palatine canal, and the electrode passes out of the greater palatine canal into the pterygopalatine fossa. Upon detection of passage into the fossa, the electrode or, if the electrode is a temporary electrode, another implantable electrode is implanted in a vicinity of the sphenopalatine ganglion (SPG) (also called the pterygopalatine ganglion). The external sensing sites are on the head of the subject, such as the face or ears. As used in the present application, including in the claims, the “distal” end of the bony canal is the far end of the canal toward which the electrode moves as the electrode is advanced through the canal. The distal end of the greater palatine canal, which is the higher end when the subject's head is upright, is sometimes referred to in the art as the “superior” end of the canal.

In some embodiments of the present invention, a neural stimulation system comprises a neural stimulator and an external electrode positioning unit. The neural stimulator comprises an elongated support element and one or more first electrodes fixed to the support element in a vicinity of a distal end thereof. The support element is configured to be advanced at least partially through a bony canal, such as a greater palatine canal. The neural stimulation system comprises one or more external second electrodes, configured to be placed at respective sensing sites on an external surface of a body of the subject. The positioning unit is configured to sense an electrical parameter at the sites using the external second electrodes, while the first electrode is electrically activated and advanced at least partially through the bony canal. For some applications, the positioning unit is configured to detect passage of the first electrode out of a distal end of the canal, such as into a pterygopalatine fossa in a vicinity of the SPG, responsively to a change in the electrical parameter, and to generate an output indicative of the detected passage. Alternatively, the positioning unit is configured to generate an output indicative of a value of the sensed electrical parameter, and a healthcare worker performing the implantation procedure detects the passage responsively to the outputted value.

In some embodiments of the present invention, the external electrode positioning unit is electrically coupled to two or more of the first electrodes of the neural stimulator. The positioning unit is configured to generally constantly sense an electrical parameter while the first electrodes are electrically activated (by driving a current between the two or more first electrodes) and advanced at least partially through the bony canal. A change in the sensed parameter indicates passage of the first electrodes out of the canal, such as into the pterygopalatine fossa in a vicinity of the SPG.

Activation or inhibition of the SPG using the electrodes is useful for treatment of various brain conditions, such as by affecting properties of the brain including cerebral blood flow (CBF), permeability of the blood-brain-barrier (BBB), and release of neurotransmitters. For some applications, techniques described herein are performed in combination with techniques described in one or more of the applications assigned to the assignee of the present application and incorporated by reference hereinbelow. The techniques of embodiments of the present invention generally enable quick and accurate identification of the proper implantation location in real time during an implantation procedure, such that the healthcare worker performing the implantation procedure is able to adjust the positioning of the electrodes as necessary. Such adjustment of the electrode positioning in real time during the implementation procedure generally reduces the likelihood that the electrode positioning will need to be adjusted in a subsequent, separate procedure. In addition, the quick placement of the electrodes enabled by the techniques of the present invention may be particularly helpful for the acute phase of treatment of conditions such as stroke, in which the timeliness of treatment often affects the clinical outcome.

For some applications, treatment with the systems described herein is applied as soon as possible after diagnosis of the condition, such as in an emergency room or wherever the subject happens to be. For other applications, the system is appropriate for longer-term treatment, such as for modulating the permeability of the BBB, modulating cerebral blood flow (CBF), rehabilitation after brain events, or prevention and/or treatment of epilepsy. For some applications, the stimulator or, if the stimulator is a temporary stimulator, another implantable stimulator is configured to be implanted for at least one week, e.g., at least one month, while for other applications, the stimulator or another implantable stimulator is adapted to be implanted for less than one week, e.g., less than one day.

There is therefore provided, in accordance with an embodiment of the present invention, apparatus including a surgical tool, which includes:

a proximal shaft;

a distal rod, a proximal end thereof which is coupled to a distal end of the proximal shaft such that the distal rod articulates with the proximal shaft; and

a target sight, which includes an aiming element, which is coupled to the distal rod and extends toward the proximal shaft, and which is indicative of an alignment of the distal rod with respect to the proximal shaft.

For some applications, the proximal shaft has a length of at least 50 mm, the distal rod has a length of between 10 and 50 mm, and a portion of the distal rod has a length of at least 5 mm and a greatest diameter of between 0.5 and 2 mm.

For some applications, the target sight further includes an aiming ring, which is coupled to the proximal shaft, and the aiming element and the aiming ring are arranged such that the proximal shaft is generally centered with respect to the aiming ring when a central longitudinal axis of the distal element is parallel to a central longitudinal axis of the proximal shaft through a distal end of the proximal shaft.

For some applications, the proximal shaft is shaped so as to define a proximal shaft bend. For some applications, the aiming element is shaped so as to define an aiming element bend having a same angle as the proximal shaft bend. For some applications, the proximal shaft is shaped so as to define the proximal shaft bend at a location along the proximal shaft between 20 and 60 mm from a distal end of the distal rod, and the proximal shaft bend has an angle of between 90 and 175 degrees.

For some applications, the distal rod and the proximal shaft are arranged to allow the distal rod to articulate with the proximal shaft in all directions. For some applications, the proximal shaft is shaped so as to define a proximal shaft bend.

Alternatively, the distal rod and the proximal shaft are arranged to allow the distal rod to articulate with the proximal shaft with greater than one degree of freedom.

There is further provided, in accordance with an embodiment of the present invention, a method including:

providing a surgical tool, which includes:
a proximal shaft,
a distal rod, a proximal end thereof which is coupled to a distal end of the proximal shaft such that the distal rod articulates with the proximal shaft, and
a target sight, which includes an aiming element, which is coupled to the distal rod and extends toward the proximal shaft, and which is indicative of an alignment of the distal rod with respect to the proximal shaft; and preparing a greater palatine canal of a subject by:
advancing the surgical tool through at least a portion of the canal, and
ascertaining the alignment using the target sight.

For some applications, preparing the canal further includes, upon ascertaining that the distal rod is not properly aligned with respect to the proximal shaft, adjusting an orientation of the proximal shaft in order to properly align the distal rod with the shaft while ascertaining the alignment using the target sight.

For some applications, the target sight further includes an aiming ring, which is coupled to the proximal shaft, the aiming element and the aiming ring are arranged such that the proximal shaft is generally centered with respect to the aiming ring when a central longitudinal axis of the distal rod is parallel to a central longitudinal axis of the proximal shaft through a distal end of the proximal shaft, and ascertaining the alignment includes observing a position of the aiming element with respect to the aiming ring.

For some applications, the distal rod and the proximal shaft are arranged to allow the distal rod to articulate with the proximal shaft in all directions. Alternatively, the distal rod and the proximal shaft are arranged to allow the distal rod to articulate with the proximal shaft with greater than one degree of freedom.

There is still further provided, in accordance with an embodiment of the present invention, apparatus including a surgical tool, which includes:

a proximal shaft; and

a distal rod, a proximal end thereof which is coupled to a distal end of the proximal shaft such that the distal rod articulates with the proximal shaft,

wherein the surgical tool is configured to allow the rod to articulate with the proximal shaft with exactly one degree of freedom, such that the rod, as it articulates with the one degree of freedom, defines a plane,

wherein a first portion of the distal rod facing in a first direction perpendicular to the plane is shaped so as to define an abrading surface, and a second portion of the distal rod facing in a second direction opposite the first direction is not shaped so as to define an abrading surface.

For some applications, the proximal shaft is shaped so as to define a bend (e.g., having an angle of between 90 and 175 degrees), which bend includes a radially outward bend portion and a radially inward bend portion, and the first portion of the distal rod that is shaped so as to define the abrading surface faces generally in the same direction that the radially outward bend portion faces.

For some applications, the surgical tool further includes a resisting element, which is arranged to resist articulation of the distal rod with the proximal shaft, and the distal rod, the proximal shaft, and the resisting element are arranged to allow the rod to articulate with the proximal shaft with the exactly one degree of freedom. For some applications, the resisting element is flat, such that it articulates with the exactly one degree of freedom. Alternatively or additionally, the resisting element couples the distal end of the proximal shaft to the proximal end of the distal rod. Further alternatively or additionally, the resisting element includes an elastic element.

There is additionally provided, in accordance with an embodiment of the present invention, a method including:

providing at least one surgical tool, which includes:
a proximal shaft; and
a distal rod, a proximal end thereof which is coupled to a distal end of the proximal shaft such that the distal rod articulates with the proximal shaft,
wherein the surgical tool is configured to allow the rod to articulate with the proximal shaft with exactly one degree of freedom such that the rod, as it articulates with the one degree of freedom, defines a plane,
wherein a first portion of the distal rod facing in a first direction perpendicular to the plane is shaped so as to define an abrading surface, and a second portion of the distal rod facing in a second direction opposite the first direction is not shaped so as to define an abrading surface; and preparing a greater palatine canal of a subject by:
advancing the surgical tool through at least a portion of the canal such that a distal tip of the distal rod applies a forward longitudinal force in order to open a passage through the canal, and
using the abrading surface to abrade a posterior wall of the canal.

For some applications, the proximal shaft is shaped so as to define a bend (e.g., having an angle of between 90 and 175 degrees), which bend includes a radially outward bend portion and a radially inward bend portion, and the first portion of the distal rod that is shaped so as to define the abrading surface faces generally in the same direction that the radially outward bend portion faces, and providing the at least one surgical tool includes providing the at least one surgical tool shaped so as to define the bend.

For some applications, the surgical tool further includes a resisting element, which is arranged to resist articulation of the distal rod with the proximal shaft, the distal rod, the proximal shaft, and the resisting element are arranged to allow the rod to articulate with the proximal shaft with the exactly one degree of freedom, and providing the at least one surgical tool includes providing the at least one surgical tool including the resisting element. For some applications, the resisting element is elastic, and providing the at least one surgical tool includes providing the at least one surgical tool including the elastic element.

There is yet additionally provided, in accordance with an embodiment of the present invention, apparatus including a surgical tool, which includes:

a proximal shaft, which has a length of at least 50 mm;

a distal rod, a proximal end thereof which is coupled to a distal end of the proximal shaft such that the distal rod articulates with the proximal shaft, which distal rod has a length of between 10 and 50 mm, and includes a portion that has a length of at least 5 mm and a greatest diameter of between 0.5 and 2 mm; and

a resisting element, which is arranged to resist articulation of the distal rod with the proximal shaft.

For some applications, the resisting element couples the distal end of the proximal shaft to the proximal end of the distal rod.

Alternatively or additionally, the resisting element includes an elastic element.

For some applications, the proximal shaft is shaped so as to define a bend. For some applications, the proximal shaft is shaped so as to define the bend at a location along the proximal shaft between 20 and 60 mm from a distal tip of the distal rod, and the proximal shaft bend has an angle of between 90 and 175 degrees.

For some applications, the distal rod is shaped so as to define a blunt distal tip.

For some applications, when the distal rod articulates with the proximal shaft, a central longitudinal rod axis of the distal rod defines an angle with a central longitudinal shaft axis of the proximal shaft through the distal end of the proximal shaft, and the tool further includes an articulation limiting element that is configured to limit the angle to a maximum angle. For some applications, the maximum angle has a value that is no more than 10 degrees.

For some applications, the distal rod, the proximal shaft, and the resisting element are arranged to allow the rod to articulate with the proximal shaft with greater than one degree of freedom.

For some applications, the distal rod, the proximal shaft, and the resisting element are arranged to allow the rod to articulate with the proximal shaft in all directions. For some applications, the resisting element includes a flexible rod. For some applications, the distal rod is shaped so as to define a hollow area within 20 mm of the proximal end thereof, and at least a portion of the resisting element is positioned within the hollow area. For some applications, an external wall of the hollow area is shaped so as to define at least one opening therethrough.

For some applications, the distal rod, the proximal shaft, and the resisting element are arranged to allow the rod to articulate with the proximal shaft with exactly one degree of freedom. For some applications, the rod, as it articulates with the one degree of freedom, defines a plane, a first portion of the distal rod facing in a first direction perpendicular to the plane is shaped so as to define an abrading surface, and a second portion of the distal rod facing in a second direction opposite the first direction is not shaped so as to define an abrading surface. For some applications, the proximal shaft is shaped so as to define a bend, which bend includes a radially outward bend portion and a radially inward bend portion, and the first portion of the distal rod that is shaped so as to define the abrading surface faces generally in the same direction that the radially outward bend portion faces. For some applications, the proximal shaft is shaped so as to define the bend at a location along the proximal shaft between 20 and 60 mm from a distal tip of the distal rod, and the proximal shaft bend has an angle of between 90 and 175 degrees.

For some applications, the resisting element is flat, such that it articulates with the exactly one degree of freedom.

For some applications, the surgical tool further includes a target sight, which includes an aiming element, which is coupled to the distal rod and extends toward the proximal shaft, and which is indicative of an alignment of the distal rod with respect to the proximal shaft. For some applications, the target sight further includes an aiming ring, which is coupled to the proximal shaft, and the aiming element and the aiming ring are arranged such that the proximal shaft is generally centered with respect to the aiming ring when a central longitudinal axis of the distal rod is parallel to a central longitudinal axis of the proximal shaft through a distal end of the proximal shaft.

There is also provided, in accordance with an embodiment of the present invention, apparatus including a surgical kit, which includes:

at least first and second surgical tools, each of which includes:
a proximal shaft;
a distal rod, a proximal end thereof which is coupled to a distal end of the proximal shaft such that the distal rod articulates with the proximal shaft; and
a resisting element, which is arranged to resist articulation of the distal rod with the proximal shaft,

wherein the distal rod, the proximal shaft, and the resisting element of the first surgical are arranged to allow the rod to articulate with the proximal shaft with greater than one degree of freedom, and

wherein the distal rod, the proximal shaft, and the resisting element of the second surgical tool are arranged to allow the rod to articulate with the proximal shaft with exactly one degree of freedom; the rod, as it articulates with the one degree of freedom, defines a plane; a first portion of the distal rod of the second surgical tool facing in a first direction perpendicular to the plane is shaped so as to define an abrading surface; and a second portion of the distal rod facing in a second direction opposite the first direction is not shaped so as to define an abrading surface.

For some applications, the proximal shafts of the first and second surgical tools have respective lengths of at least 50 mm, and the distal rods of the first and second surgical tools have respective lengths of between 10 and 50 mm, and include respective portions that have respective lengths of at least 5 mm and respective greatest diameters of between 0.5 and 2 mm.

For some applications, the distal rod of at least one of the first and second surgical tools is shaped so as to define a blunt distal tip.

For some applications, the second surgical tool includes at least the second surgical tool and a third surgical tool, and a diameter of the first portion defining the abrading surface of the third surgical tool is greater than a diameter of the first portion defining the abrading surface of the second surgical tool.

There is further provided, in accordance with an embodiment of the present invention, a method including:

providing at least one surgical tool, which includes:
a proximal shaft;
a distal rod, a proximal end thereof which is coupled to a distal end of the proximal shaft such that the distal rod articulates with the proximal shaft; and
a resisting element, which is arranged to resist articulation of the distal rod with the proximal shaft; and

preparing a greater palatine canal of a subject by advancing the surgical tool through at least a portion of the canal such that a distal tip of the distal rod applies a forward longitudinal force in order to open a passage through the canal.

For some applications, the resisting element is elastic, and providing the at least one surgical tool includes providing the at least one surgical tool including the elastic element.

For some applications, the distal rod, the proximal shaft, and the resisting element are arranged to allow the rod to articulate with the proximal shaft in all directions, and advancing includes advancing the surgical tool through the at least the portion of the canal substantially without applying forces to walls of the canal in directions perpendicular to a longitudinal axis of the rod.

For some applications, the distal rod, the proximal shaft, and the resisting element are arranged to allow the rod to articulate with the proximal shaft with greater than one degree of freedom, and advancing includes advancing the surgical tool through the at least the portion of the canal substantially without applying forces to walls of the canal in directions perpendicular to a longitudinal axis of the rod.

For some applications, the distal rod, the proximal shaft, and the resisting element are arranged to allow the rod to articulate with the proximal shaft with exactly one degree of freedom; the rod, as it articulates with the one degree of freedom, defines a plane; a first portion of the distal rod facing in a first direction perpendicular to the plane is shaped so as to define an abrading surface; and a second portion of the distal rod facing in a second direction opposite the first direction is not shaped so as to define an abrading surface, and advancing includes using the abrading surface to abrade a posterior wall of the canal.

For some applications, advancing the surgical tool includes orienting the proximal shaft of the surgical tool using a target sight, which includes: (a) an aiming element, which is coupled to the distal rod and extends toward the proximal shaft, and which is indicative of an alignment of the distal rod with respect to the proximal shaft, and (b) an aiming ring, which is coupled to the proximal shaft, and orienting the proximal shaft includes observing a position of the aiming element with respect to the aiming ring.

For some applications, the method further includes, after advancing the surgical tool, entirely withdrawing the surgical tool from the canal, and inserting a neural stimulator into the prepared canal.

There is still further provided, in accordance with an embodiment of the present invention, a method including:

electrically activating at least one electrode;

sensing an electrical parameter while the electrode is advanced at least partially through a greater palatine canal of a subject and while the electrode is electrically activated; and

generating an output responsively to the electrical parameter.

For some applications, generating the output includes generating the output indicative of a value of the electrical parameter.

For some applications, generating the output includes performing an analysis of the electrical parameter, and generating the output indicative of the analysis.

For some applications, the method further includes detecting passage of the electrode out of a distal end of the canal to a pterygopalatine fossa responsively to a change in a value of the electrical parameter. For some applications, the method further includes ascertaining an implantation location responsively to the detected passage, and implanting the electrode at the implantation location.

For some applications, the at least one electrode is at least one temporary first electrode, and the method further includes: ascertaining an implantation location responsively to the detected passage; withdrawing the temporary first electrode from the bony canal; and implanting an implantable second electrode at the ascertained implantation location. For some applications, the electrical parameter is selected from the group consisting of: an impedance, a voltage, and a current, and detecting the passage includes detecting the passage responsively to the change in the selected parameter.

For some applications, electrically activating the electrode includes wirelessly transmitting energy to the electrode from outside the body.

For some applications, sensing includes sensing the electrical parameter at one or more sensing sites on an external surface of a body of the subject. For some applications, the at least one electrode is at least one first electrode, and sensing the electrical parameter includes: driving a current between the at least one first electrode and one or more external second electrodes placed at respective ones of the sensing sites, which external second electrodes are coupled to the first electrode by at least one metal conductor; and sensing an electrical parameter of the current. For some applications, the at least one electrode is at least one first electrode, and sensing the electrical parameter includes sensing using one or more external second electrodes placed at respective ones of the sensing sites, which external second electrodes are not coupled to the first electrode by any metal conductors. For some applications, the external surface is skin of a face of the subject, and sensing including sensing at the one or more sensing sites on the skin of the face.

For some applications, electrically activating includes electrically activating the electrode using a first control unit, and sensing includes sensing the electrical parameter using a second control unit that is not operatively coupled to the first control unit.

For some applications, the at least one electrode is coupled to a neural stimulator, and sensing includes sensing the parameter while the neural stimulator is advanced at least partially through the bony canal, and the method further includes:

mechanically measuring a distance that the neural stimulator is advanced at least partially through the bony canal;

comparing the measured distance to a threshold value based on a length of the bony canal; and

detecting passage of the electrode out of a distal end of the canal responsively to a change in the electrical parameter, only if the measured distance exceeds the threshold value.

For some applications, the at least one electrode includes two or more electrodes, electrically activating includes driving a current between the two or more electrodes, and sensing includes sensing the electrical parameter of the current as the two or more electrodes are advanced at least partially through the greater palatine canal and while the current is driven between the electrodes. For some applications, the electrodes are coupled to an introducer tool, and sensing includes sensing the parameter as the introducer tool is advanced at least partially through the bony canal.

For some applications, sensing includes sensing the electrical parameter at one or more sensing sites in a cavity of a body of the subject, the cavity selected from the group consisting of: an oral cavity and a nasal cavity.

There is additionally provided, in accordance with an embodiment of the present invention, apparatus including:

an elongated support element configured to be advanced at least partially through a greater palatine canal of a subject;

at least one first electrode fixed to the support element in a vicinity of a distal end thereof, and configured to be electrically activated; and

an external electrode positioning unit, including:
a driving unit, configured to activate the at least one first electrode;
a sensing unit, configured to sense an electrical parameter while the first electrode is electrically activated and advanced at least partially through the canal; and
an output unit, configured to generate an output responsively to the electrical parameter.

For some applications, the support element has a length of between 1.8 cm and 4 cm.

For some applications, the output is configured to generate the output indicative of a value of the electrical parameter.

For some applications, the external electrode positioning unit further includes an analysis unit, and the output unit is configured to generate the output indicative of the analysis. For some applications, the analysis unit is configured to detect passage of the electrode out of a distal end of the canal responsively to a change in the electrical parameter, and the output unit is configured to generate the output indicative of the detected passage. For some applications, the electrical parameter is selected from the group consisting of: an impedance, a voltage, and a current, and the analysis unit is configured to detect the passage responsively to the change in the selected parameter.

For some applications, the electrode positioning unit is configured to electrically activate the first electrode.

For some applications, the apparatus further includes an external wireless energy transmitter, which is configured to be placed outside the body of the subject, and to wirelessly transmit energy to the first electrode for activating the first electrode.

For some applications, the apparatus further includes a control unit which is not operatively coupled to the electrode positioning unit, and which is configured to electrically activate the first electrode.

For some applications, the external electrode positioning unit includes one or more external second electrodes, configured to be placed at respective sensing sites on an external surface of a body of the subject, and the sensing unit is configured to sense the electrical parameter at the sites using the one or more external second electrodes while the first electrode is electrically activated and advanced at least partially through the canal. For some applications, the apparatus further includes at least one metal conductor which couples the one or more external second electrodes to the first electrode. For some applications, the one or more external second electrodes are not coupled to the first electrode by any metal conductors.

For some applications, the at least one first electrode includes two or more first electrodes, which are configured to be advanced at least partially through the canal, the driving unit is configured to drive a current between the first electrodes, and the sensing unit is configured to sense the electrical parameter of the current while the driving unit drives the current and the first electrodes are advanced at least partially through the canal. For some applications, the apparatus further includes an introducer tool, configured to advance the first electrodes at least partially through the canal of a subject, and at least one of the first electrodes is coupled to the introducer tool in a vicinity of a distal end of the tool.

For some applications, the external electrode positioning unit includes one or more intracavitary second electrodes, configured to be placed at respective sensing sites in a cavity of a body of the subject selected from the group consisting of: an oral cavity and a nasal cavity, and the sensing unit is configured to sense the electrical parameter at the sites using the one or more intracavitary second electrodes while the first electrode is electrically activated and advanced at least partially through the canal.

There is yet additionally provided, in accordance with an embodiment of the present invention, a method including:

advancing a position assessment element into a body of a subject in a vicinity of a greater palatine canal;

activating the position assessment element to emit energy;

sensing the emitted energy;

analyzing the sensed energy; and

responsively to the analyzing, ascertaining whether the position assessment unit is within or outside the bony canal.

For some applications, the method further includes generating an output indicative of whether the position assessment element is within or outside the greater palatine canal.

For some applications, the position assessment element includes at least one ultrasound transducer, and activating includes activating the at least one ultrasound transducer to emit ultrasonic energy. For some applications, analyzing the sensed ultrasonic energy includes analyzing the sensed ultrasonic energy to find a density of tissue within a certain distance of the at least one transducer, and ascertaining includes ascertaining responsively to the density and the distance. For example, the certain distance may be between 0.5 and 5 mm.

For some applications, the method further includes generating an output indicative of the density, and ascertaining includes receiving the output, and ascertaining responsively to the output.

For some applications, analyzing the ultrasonic energy includes generating an ultrasound image of an area near the at least one transducer, and analyzing the image to find the density of the tissue near the at least one transducer.

For some applications, advancing the position assessment element includes advancing, along an advancement route, a shaft to which the position assessment element is coupled, and the method further includes withdrawing the shaft from the body after ascertaining that the position assessment element is within the greater palatine canal, and subsequently advancing a neural stimulator along the advancement route into the greater palatine canal.

For some applications, the position assessment element includes a light-emitting element, activating the position assessment element to emit the energy includes activating the light-emitting element to emit light, and sensing the sensed energy includes sensing the sensed light from a site selected from the group consisting of: a site within a mouth of the subject, a site within a nose of the subject, and a site external to a face of the subject.

For some applications, sensing includes sensing the light from within the mouth. Alternatively, analyzing includes analyzing a level of illumination of an external surface of the face.

For some applications, analyzing the sensed light includes detecting an intensity of the light, and ascertaining includes ascertaining responsively to the intensity. For some applications, the method further includes generating an output indicative of the intensity, and ascertaining includes receiving the output, and ascertaining responsively to the output. For some applications, detecting the intensity includes detecting the intensity using a light sensor. Alternatively or additionally, sensing the emitted light includes viewing the emitted light by a healthcare worker.

For some applications, activating the light-emitting element includes activating the light-emitting element to emit the light intermittently.

There is also provided, in accordance with an embodiment of the present invention, a method including:

advancing a balloon into a body of a subject in a vicinity of a greater palatine canal;

at least partially inflating the balloon with a fluid having a volume, and measuring a pressure of the fluid; and

ascertaining whether the balloon is within or outside the greater palatine canal, responsively to the pressure and to at least one parameter selected from the group consisting of: the volume, and a rate of change of the volume.

For some applications, ascertaining includes calculating a ratio of the volume to the pressure, and ascertaining responsively to the ratio.

For some applications, the method further includes generating an output indicative of whether the balloon is within or outside the greater palatine canal.

For some applications, advancing the balloon includes advancing, along an advancement route, a shaft to which the balloon is coupled, and the method further includes withdrawing the shaft from the body after ascertaining that the balloon is within the greater palatine canal, and subsequently advancing a neural stimulator along the advancement route into the greater palatine canal.

There is further provided, in accordance with an embodiment of the present invention, a method including:

advancing, into a body of a subject in a vicinity of a greater palatine canal, a shaft having an opening that is in fluid communication with a fluid source via a channel;

injecting a fluid having a volume, from the fluid source via the channel through the opening into the body, and measuring a pressure of the fluid; and

ascertaining whether the opening is within or outside the greater palatine canal, responsively to the pressure and to at least one parameter selected from the group consisting of: the volume, and a rate of change of the volume.

For some applications, ascertaining includes calculating a ratio of the volume to the pressure, and ascertaining responsively to the ratio.

For some applications, the method further includes generating an output indicative of whether the opening is within or outside the greater palatine canal.

For some applications, the method further includes withdrawing the shaft from the body after ascertaining that the opening is within the greater palatine canal, and subsequently advancing a neural stimulator along the advancement route into the greater palatine canal.

There is still further provided, in accordance with an embodiment of the present invention, apparatus for advancement into a body of a subject in a vicinity of a greater palatine canal, the apparatus including:

a semi-rigid or rigid shaft;

a position assessment element which is coupled to the shaft, and which is configured to emit energy;

a position assessment unit, which is configured to:
analyze the energy emitted by the position assessment unit, and
responsively to the analysis, ascertain whether the position assessment unit is within or outside the greater palatine canal; and

an output unit, configured to generate an output indicative of whether the position assessment unit is within or outside the greater palatine canal.

For some applications, the shaft has a length of between 15 and 40 mm, and a greatest diameter of less than 2 mm, and the position assessment element is coupled to the shaft within 10 mm a distal end of the shaft.

For some applications, the position assessment element includes at least one ultrasound transducer, which is configured to emit ultrasonic energy. For some applications, the position assessment unit is configured to analyze the emitted ultrasonic energy to find a density of tissue within a certain distance of the at least one transducer, and, responsively to the density and the distance, ascertain whether the at least one transducer is within or outside the greater palatine canal. For example, the certain distance may be between 0.5 and 5 mm.

For some applications, the shaft is shaped so as to define a bend having an angle of between 150 and 170 degrees.

For some applications, the apparatus further includes a neural stimulator that includes: a stimulator shaft having a length of between 15 and 40 mm, and a greatest diameter of less than 2 mm; and at least one electrode coupled to the stimulator shaft.

For some applications, the position assessment element includes a light-emitting element, which is configured to emit light; the position assessment unit includes a light sensor, which is configured to detect an intensity of the emitted light; and the position assessment unit is configured to ascertain, responsively to the intensity, whether the light-emitting element is within or outside the greater palatine canal. For some applications, the light-emitting element is configured to emit the light intermittently.

There is additionally provided, in accordance with an embodiment of the present invention, apparatus including:

a semi-rigid or rigid shaft;

a balloon coupled to the shaft;

a fluid source in fluid communication with the balloon, which fluid source is configured to contain a fluid, and to at least partially inflate the balloon with a volume of the fluid;

a pressure sensor, which is configured to measure a pressure of the fluid when the balloon is at least partially inflated;

a position assessment unit, which is configured to ascertain whether the balloon is within or outside a greater palatine canal, responsively to the pressure and to at least one parameter selected from the group consisting of: the volume, and a rate of change of the volume; and

an output unit, configured to generate an output indicative of whether the balloon is within or outside the greater palatine canal.

For some applications, the shaft is shaped so as to define a bend having an angle of between 150 and 170 degrees.

For some applications, the position assessment unit is configured to calculate a ratio of the volume to the pressure, and to ascertain whether the balloon is within or outside the greater palatine canal responsively to the ratio.

For some applications, the apparatus further includes a neural stimulator that includes: a stimulator shaft having a length of between 15 and 40 mm, and a greatest diameter of less than 2 mm; and at least one electrode coupled to the stimulator shaft.

For some applications, the shaft has a length of between 15 and 40 mm, and a greatest diameter of less than 2 mm, and the balloon is coupled to the shaft within 10 mm of a distal end of the shaft.

There is yet additionally provided, in accordance with an embodiment of the present invention, apparatus including:

a semi-rigid or rigid shaft shaped so as to define an opening and a channel in fluid communication with the opening;

a fluid source in fluid communication with the opening via the channel, which fluid source is configured to contain a fluid, and to inject the fluid having a volume via the channel through the opening;

a pressure sensor, which is configured to measure a pressure of the injected fluid, and to generate a signal indicative of the pressure;

a position assessment unit, which is configured to:
receive the signal, and
ascertain whether the opening is within or outside a greater palatine canal, responsively to the pressure and to at least one parameter selected from the group consisting of: the volume, and a rate of change of the volume; and

an output unit, configured to generate an output indicative of whether the opening is within or outside the greater palatine canal.

For some applications, the position assessment unit is configured to calculate a ratio of the volume to the pressure, and to ascertain whether the opening is within or outside the greater palatine canal responsively to the ratio.

For some applications, the shaft is shaped so as to define a bend having an angle of between 150 and 170 degrees.

For some applications, the apparatus further includes a neural stimulator that includes: a stimulator shaft having a length of between 15 and 40 mm, and a greatest diameter of less than 2 mm; and at least one electrode coupled to the stimulator shaft.

For some applications, the shaft has a length of between 15 and 40 mm, and a greatest diameter of less than 2 mm.

The present invention will be more fully understood from the following detailed description of the embodiments thereof, taken together with the drawings in which:

### BRIEF DESCRIPTION OF THE DRAWINGS

FIG. 1 is a schematic illustration of a forward tool for preparing a bony canal, in accordance with an embodiment of the present invention;

FIGS. 2A-B are schematic cross-sectional illustrations of a portion of the forward tool of FIG. 1, showing the articulation of a distal rod with a proximal shaft of the tool, in accordance with an embodiment of the present invention;

FIG. 3 is a schematic illustration of an abrading tool for opening a passage through and abrading a bony canal, in accordance with an embodiment of the present invention;

FIGS. 4A-B are schematic illustrations of the abrading tool of FIG. 3, showing the articulation of a distal rod with a proximal shaft of the tool, in accordance with an embodiment of the present invention;

FIGS. 5A-B are schematic illustrations of steps of a canal preparation procedure performed on a subject using the tools of FIGS. 1 and 3, in accordance with an embodiment of the present invention;

FIG. 6 is a flow chart schematically illustrating the steps of the procedure of FIGS. 5A-B, in accordance with an embodiment of the present invention;

FIG. 7 is a schematic illustration of an introducer tool and a neural stimulator, in accordance with an embodiment of the present invention;

FIGS. 8A-C are schematic illustrations of a target sight applied to the forward tool of FIG. 1, in accordance with an embodiment of the present invention;

FIG. 9 is a schematic illustration of a neural stimulation system applied to a subject, shown in frontal view, in accordance with an embodiment of the present invention;

FIG. 10 is a schematic illustration of the neural stimulation system of FIG. 1 applied to the subject, shown in cross-sectional view, in accordance with an embodiment of the present invention;

FIG. 11 is a schematic illustration of an alternative configuration of the neural stimulation system of FIGS. 1 and 2, in accordance with an embodiment of the present invention;

FIG. 12 is a schematic illustration of an introducer tool for implanting a neural stimulator, in accordance with an embodiment of the present invention;

FIGS. 13A and 13B are schematic illustrations of a probe system applied to a subject, shown in cross-sectional view, in accordance with an embodiment of the present invention;

FIG. 14 which is a schematic illustration of a guidance jig, in accordance with an embodiment of the present invention;

FIGS. 15A-B are schematic illustrations an ultrasound probe, in accordance with respective embodiments of the present invention;

FIG. 16 is a schematic illustration a light-emitting probe, in accordance with an embodiment of the present invention;

FIG. 17 is a schematic illustration a balloon probe, in accordance with an embodiment of the present invention;

FIG. 18 is a graph showing two pressure-volume curves, in accordance with an embodiment of the present invention; and

FIG. 19 is a schematic illustration of a fluid injection probe, in accordance with an embodiment of the present invention.

### DETAILED DESCRIPTION OF EMBODIMENTS

FIG. 1 is a schematic illustration of a forward tool 20 for preparing a bony canal, in accordance with an embodiment of the present invention. Forward tool 20 is configured to open a passage through the canal by applying a forward longitudinal force, schematically indicated by an arrow 22, substantially without applying forces to the walls of the canal in directions perpendicular to a central longitudinal rod axis 60 of distal rod 30. These directions are schematically indicated by arrows 24 in FIG. 1. The tool opens the passage through soft tissue within the canal, and/or between anatomical features of the canal, such as between soft tissue and the osseous wall of the canal.

Forward tool 20 comprises a distal rod 30, a proximal shaft 32, and a resisting element 34. Distal rod 30 is shaped so as to define a blunt distal tip 40 that opens the passage through the canal as the tip is advanced distally through the canal and applies a forward longitudinal force. For some applications, a proximal portion of proximal shaft 32 comprises or is shaped so as to define a handle 42, which typically has a greater diameter than the diameter of a more distal portion 47 of proximal shaft 32. For example, the handle may have a length of 10 cm. Typically, distal rod 30 and proximal shaft 32 comprise a substantially rigid material, such as a metal, e.g., steel. For some applications, distal portion 47 of proximal shaft 32 comprises stainless steel, while handle 42 of proximal shaft 32 comprises aluminum.

A proximal end of distal rod 30 is coupled to a distal end of proximal shaft 32 such that distal rod 30 articulates with greater than one degree of freedom, such as in all directions (i.e., 360 degrees), as shown in the blow-up in FIG. 1. Resisting element 34 is arranged to resist articulation of distal rod 30 with proximal shaft 32. The articulation provides flexibility in several or all directions, thereby minimizing the application of forces in directions 24 to the walls of the canal, such as if proximal shaft 32 should become slightly misaligned with the direction of the canal.

In an embodiment of the present invention, resisting element 34 couples the distal end of proximal shaft 32 to the proximal end of distal rod 30, as shown in FIG. 1. Resisting element 34 typically comprises an elastic element. For some applications, resisting element 34 comprises a flexible rod. The flexible rod is short enough to support the forward application of force by distal rod 20 without buckling, while long enough to have sufficient flexibility to allow sufficient articulation of distal rod 20 with proximal shaft 32. For example, the flexible rod may have a length of between 2 and 15 mm, such as between 6 and 10 mm, e.g., 8 mm. The flexible rod may be integral to distal rod 30 and/or proximal shaft 32, or may comprise a separate element fixed to the distal rod and/or proximal shaft, e.g., by soldering or welding. For some applications, the resisting element comprises a superelastic material, such as Nitinol. Alternatively, resisting element 34 is not elastic.

Alternatively, resisting element 34 does not couple proximal shaft 32 to distal rod 30. Instead, the proximal shaft and the distal rod are coupled directly together at a joint, or are coupled together by a coupling element. In order to resist the articulation of the distal rod with the proximal shaft, the resisting element may be arranged to apply a force to an external surface or and/or an internal surface of the rod and/or shaft. For some applications, the resisting element is positioned remotely from the joint or coupling element, such as within proximal shaft or handle 42. For these applications, the resisting element conveys its resistive force via an intermediary element, which may comprise, for example, one or more elongated flexible elements, such as cords, wires, or strips.

In an embodiment of the present invention, proximal shaft 32 is shaped, typically at a location therealong between 20 and 60 mm from distal dip 40 of distal rod 30, e.g., 40 mm from the distal tip, so as to define a bend 46 having an angle α (alpha) of between 90 and 175 degrees, such as 160 degrees. The bend is ergonomically helpful to the surgeon performing the implantation procedure, because it enables insertion of the tool into the greater palatine canal within the constraints imposed by the maximum opening position of the mouth of the subject.

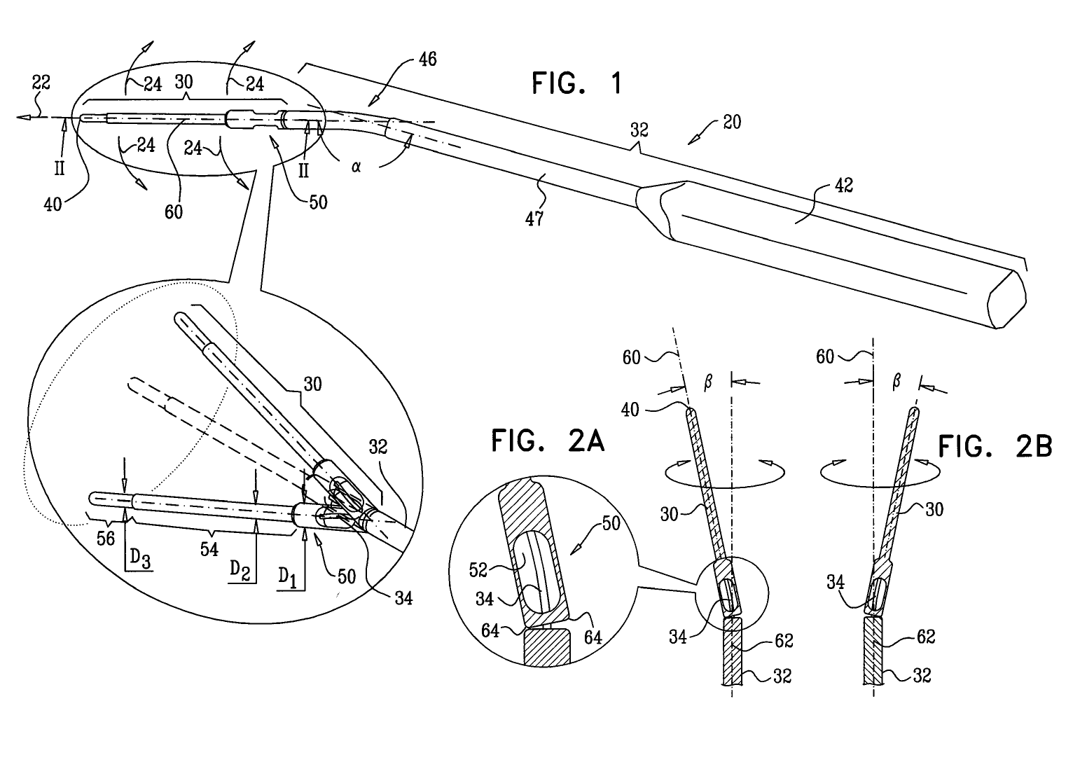

In an embodiment of the present invention, distal rod 30 is shaped so as to define a hollow area 50 within about 0 and 20 mm of the proximal end of the distal rod. At least a portion (e.g., all) of resisting element 34 is positioned within hollow area 50. This hollow area helps accommodate the resisting element within the portion of the tool distal to bend 46. The length of this portion is constrained by the combination of the angle of bend 46, the location of the bend, and the geometry of the mouth of the subject. For some applications, an external wall of hollow area 50 is shaped so as to define at least one opening 52 therethrough (shown in FIG. 2A), which may facilitate cleaning of the hollow area. Typically, hollow area 50 does not itself enter the canal during the preparation procedure.

As shown in FIG. 1, distal rod 30 typically has a greatest diameter D₁ of between 2.5 and 6 mm, such as between 3 and 5 mm, e.g., 4 mm. For some applications, the distal rod is shaped so as to define several portions having different diameters. For example, hollow area 50 may have greatest diameter D₁, an intermediary portion 54 of the rod may have a diameter D₂ that is less than D₁, e.g., between 0.5 and 2 mm, e.g., 1.3 mm, and a distal-most portion 56 of the rod may have a diameter D₃ that is less than D₂, e.g., between 0.5 and 1.5 mm, e.g., 1 mm. Typically, distal rod 30 has a length of between 10 and 50 mm, such as between 15 and 35 mm, e.g., 32 mm. Typically, intermediary portion 54 and distal-most portion 56 (i.e., all of distal rod 30 except hollow area 50) have a combined length of at least 5 mm, such as at least 10 mm or at least 15 mm, and/or between 5 and 35 mm, such as between 15 and 25 mm, e.g., 21 mm.

Typically, proximal shaft 32 (including both distal portion 47 and handle 42, if provided) has a length of at least 50 mm, such as at least 100 mm, or at least 150 mm. Typically, the length is no greater than 200 mm. For example, the length may be about 150 mm.

Typically, distal portion 47 of proximal shaft 32 has a length of at least 30 mm, such as at least 50 mm. Typically, the length is no greater than 120 mm. For example, the length may be about 50 mm.

Reference is made to FIGS. 2A-B, which are schematic cross-sectional illustrations of a portion of forward tool 20, showing the articulation of distal rod 30 with proximal shaft 32, in accordance with an embodiment of the present invention. In this embodiment, when distal rod 30 articulates with proximal shaft 32, central longitudinal rod axis 60 of distal rod 30 defines an angle β (beta) with a central longitudinal shaft axis 62 of proximal shaft 32 through the distal end of the proximal shaft. Tool 20 further comprises an articulation limiting element 64 that is configured to limit angle β to a maximum angle. For example, the maximum angle may have a value that is no more than 10 degrees, such as between 3 and 10 degrees, e.g., 5 degrees. For some applications, an outer wall of distal rod 30 at the proximal end of the rod serves as the articulation limiting element, as shown in FIG. 2A. Alternatively, a separate articulation limiting element is provided (configuration not shown).

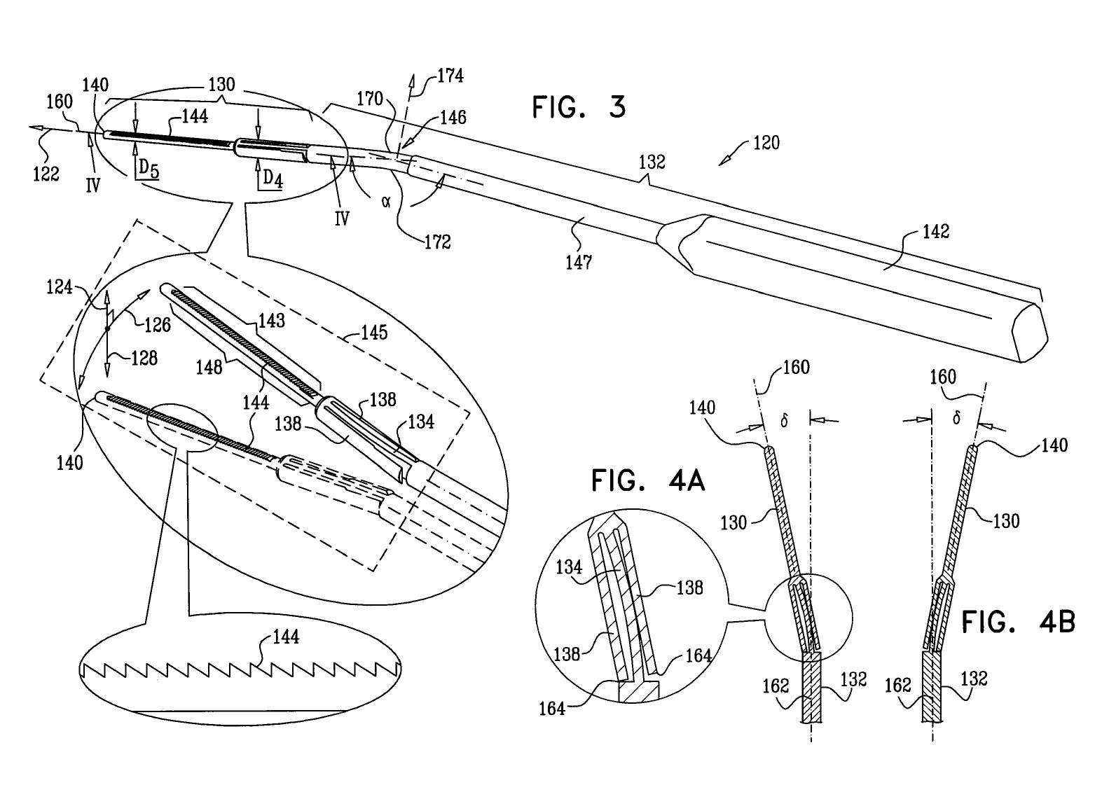

Reference is now made to FIG. 3, which is a schematic illustration of an abrading tool 120 for opening a passage through and abrading a bony canal, in accordance with an embodiment of the present invention. Abrading tool 120 is configured to open the passage through the canal by applying a forward longitudinal force, schematically indicated by an arrow 122. The abrading tool is further configured to abrade in a posterior direction, schematically indicated by an arrow 124, substantially without applying forces to the left and right walls of the canal perpendicular to the abrasion direction. The left and right directions are schematically indicated by an arrow 126 in FIG. 3.

Abrading tool 120 comprises a distal rod 130, a proximal shaft 132, and a resisting element 134. Distal rod 130 is shaped so as to define a blunt distal tip 140 that opens the passage through the canal as the tip is advanced distally through the canal and applies a forward longitudinal force. For some applications, a proximal portion of proximal shaft 132 comprises or is shaped so as to define a handle 142, which typically has a greater diameter than the diameter of a more distal portion 147 of proximal shaft 142. Typically, distal rod 130 and proximal shaft 132 comprise a substantially rigid material, such as a metal, e.g., steel. For some applications, distal portion 147 of proximal shaft 132 comprises stainless steel, while handle 142 of proximal shaft 132 comprises aluminum.

A proximal end of distal rod 130 is coupled to a distal end of proximal shaft 132 such that distal rod 130 articulates with the proximal shaft with exactly one degree of freedom. As a result, the distal rod articulates in left and right directions 126, and is prevented from articulating in posterior direction 124 and an anterior direction 128 perpendicular to two directions 126. Resisting element 134 is arranged to resist articulation of distal rod 130 with proximal shaft 132.

A first portion 143 of distal rod 130 facing in posterior direction 124 is shaped so as to define an abrading surface 144. In other words, the distal rod, as it articulates with the one degree of freedom, defines a plane 145, and portion 143 of distal rod 130 faces in direction 124 which is perpendicular to plane 145. Typically, abrading surface 144 has a length of between 5 and 25 mm (e.g., 18 mm), and a width of between 1 and 3 mm (e.g., 1.8 mm). For some applications, abrading surface 144 is shaped so as to define file-like teeth, as shown in the blow-up in FIG. 3. This arrangement provides rigidity in posterior direction 124 for abrading, while providing flexibility in left and right directions 126, thereby minimizing the application of forces to the left and right walls of the canal, such as if proximal shaft 132 should become slightly misaligned with the direction of the canal. Typically, a second portion 148 of distal rod facing in anterior direction 128 opposite posterior direction 124 is not shaped so as to define an abrading surface. In addition, at least portions of the distal rod facing in left and right directions 126 typically are not shaped so as to define abrading surfaces.

In an embodiment of the present invention, resisting element 134 couples the distal end of proximal shaft 132 to the proximal end of distal rod 130. Resisting element 134 typically comprises an elastic element. For some applications, resisting element 134 is generally flat, such that it is flexible only in the exactly two directions 126. The flat resisting element may have, for example, a thickness of between 0.1 and 0.6 mm, e.g., 0.45 mm, for example for applications in which the resisting element comprises steel. The flat element may be generally rectangular. The flat resisting element is short enough to support the forward application of force by distal rod 130 without buckling, while long enough to have sufficient flexibility to allow sufficient articulation of distal rod 130 with proximal shaft 132. For example, the flexible element may have a length of between 3 and 20, such as between 5 and 15 mm, e.g., 10 mm, for example for applications in which the resisting element comprises steel. The flexible element may be integral to distal rod 130 and/or proximal shaft 132, or may comprise a separate element fixed to the distal rod and/or proximal shaft, e.g., by soldering or welding. For some applications, the resisting element comprises a superelastic material, such as Nitinol.

Alternatively, resisting element 134 does not couple proximal shaft 132 to distal rod 130. Instead, the proximal shaft and distal rod are coupled directly together at a joint, or are coupled together by a coupling element. In order to resist the articulation of the distal rod with the proximal, the resisting element may be arranged to apply a force to an external surface or and/or an internal surface of the rod and/or shaft. For some applications, the resisting element is positioned remotely from the joint or coupling element, such as within proximal shaft or handle 142. For these applications, the resisting element conveys its resistive force via an intermediary element, which may comprise, for example, one or more elongated flexible elements, such as cords, wires, or strips.

In an embodiment of the present invention, proximal shaft 132 is shaped, typically at a location therealong between 20 and 60 mm from distal tip 140 of distal rod 130, e.g., 40 mm from the distal tip, so as to define a bend 146 having an angle α (alpha) of between 90 and 175 degrees, such as 160 degrees. The bend is ergonomically helpful to the surgeon performing the implantation procedure, because it enables insertion of the tool into the greater palatine canal within the constraints imposed by the maximum opening position of the mouth of the subject. Typically, bend 146 includes radially outward bend portion 170 and radially inward bend portion 172, and the portion of distal rod 130 that is shaped so as to define abrading surface 144 faces generally in the same direction 174 that radially outward bend portion 170 faces, i.e., within 30 degrees of direction 174.

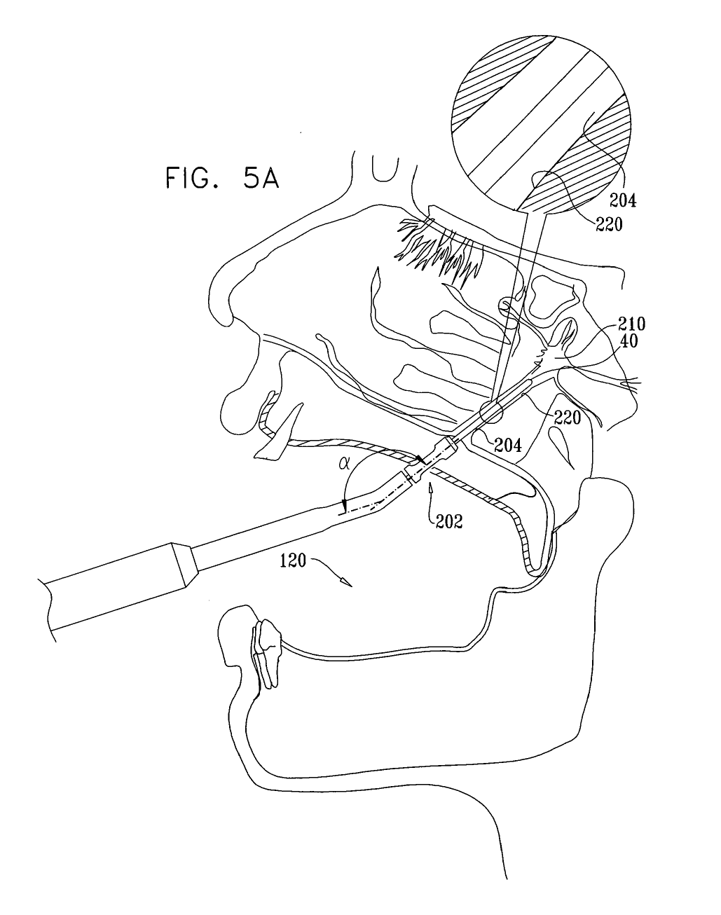

Reference is made to FIGS. 4A-B, which are schematic illustrations of abrading tool 120, showing the articulation of distal rod 130 with proximal shaft 132, in accordance with an embodiment of the present invention. In this embodiment, a proximal portion of distal rod 130 is shaped so as to define one or more (e.g., exactly two) wings 138, which flank resisting element 134. When distal rod 130 articulates with proximal shaft 132, a central longitudinal rod axis 160 of distal rod 130 defines an angle δ (delta) with a central longitudinal shaft axis 162 of proximal shaft 132 through the distal end of the proximal shaft. Tool 120 further comprises an articulation limiting element 164 that is configured to limit angle δ to a maximum angle. For example, the maximum angle may have a value that is no more than 10 degrees, such as between 3 and 10 degrees, e.g., 5 degrees. For some applications, outer walls of wings 138 at the proximal end of the rod serve as the articulation limiting element, as shown in FIG. 4A. Alternatively, a separate articulation limiting element is provided. Typically, the portion of distal rod 130 that includes wings 138 does not itself enter the canal during the preparation procedure.

Reference is again made to FIG. 3. Typically, distal rod 130 has a greatest diameter D₄ of between 2.5 and 6 mm, such as between 3 and 5 mm, e.g., 4 mm. For some applications, the distal rod is shaped so as to define several portions having different diameters. For example, the proximal portion including wings 138 may have greatest diameter D₄, and portion 143 of the distal rod that defines abrading surface 144 may have a diameter D₅ that is less than D₄, e.g., between 1.3 and 2.5 mm, such as between 1.4 and 2.1 mm, e.g., 1.5 mm. Typically, distal rod 130 has a length of between 10 and 50 mm, such as between 15 and 35 mm, e.g., 32 mm.

Typically, proximal shaft 132 (including both distal portion 147 and handle 142, if provided) has a length of at least 50 mm, such as at least 100 mm, or at least 150 mm. Typically, the length is no greater than 200 mm. For example, the length may be about 150 mm. Typically, portion 143 has a length of between 5 and 35 mm, such as between 15 and 25 mm, e.g., 21 mm.

Typically, distal portion 147 of proximal shaft 132 has a length of at least 30 mm, such as at least 50 mm. Typically, the length is no greater than 120 mm. For example, the length may be about 50 mm.

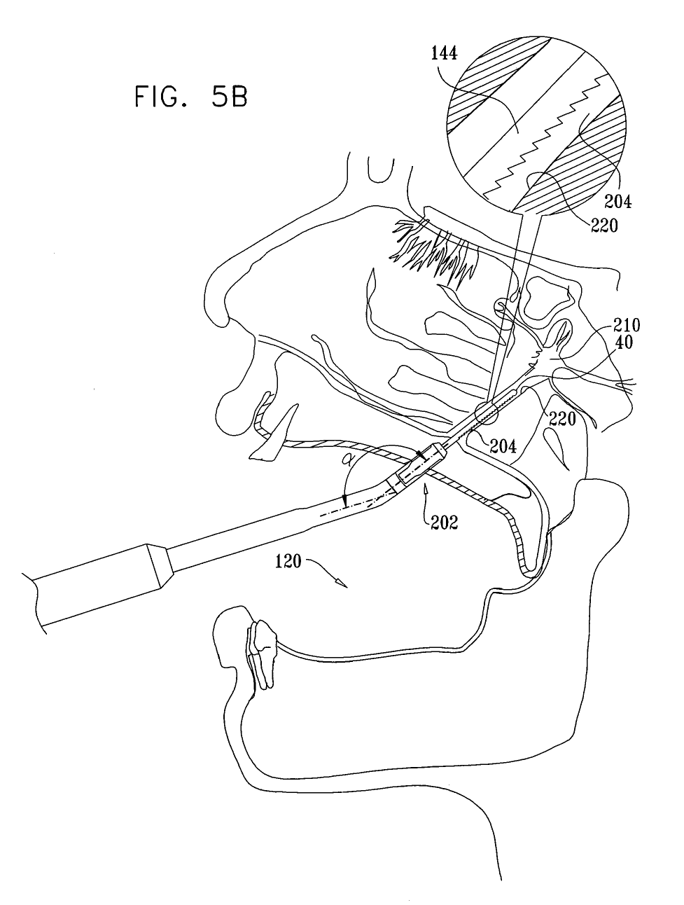

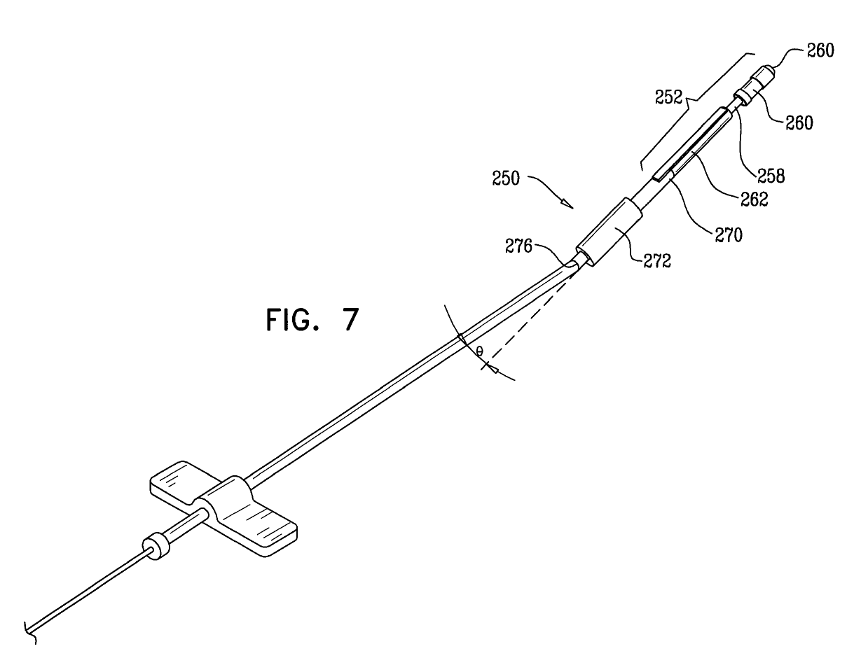

In an embodiment of the present invention, a surgical kit is provided, which comprises forward tool 20 and a plurality of abrading tools 120, such as at least two or three abrading tools. Typically, the diameters D₅ of portions 143 of abrading tools 120 are successively greater. For example, the respective diameters D₅ of abrading tools 120 may be 1.5 mm, 1.8 mm, and 2.0 mm. The abrading tools are used successively to dilate the canal, as described hereinbelow with reference to FIGS. 5A-B and 6. For some applications, the surgical kit further comprises a neural stimulator, such as described hereinbelow with reference to FIG. 7.

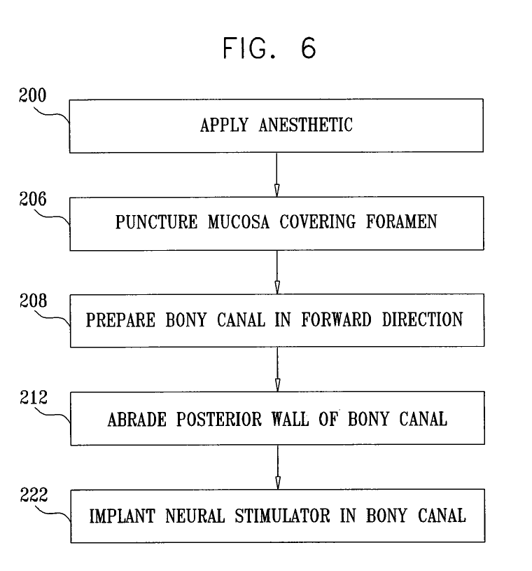

Reference is now made to FIGS. 5A-B, which are schematic illustrations of steps of a canal preparation procedure performed on a subject using tools 20 and 120, and FIG. 6, which is a flow chart schematically illustrating the steps of the procedure, in accordance with an embodiment of the present invention.

Prior to beginning the surgical procedure, the subject is typically instructed to rinse his mouth with an antimicrobial oral rinse, such as 0.2% chlorhexidine solution. For some subjects, the surgical procedure is performed under anesthesia, typically local. The procedure begins at an anesthetic step 200, at which the subject is positioned with an open mouth (typically using a mouth gag), and a topical and local anesthetic is applied to the oral palatine mucosa, such as 2 ml lidocaine. A greater palatine foramen 202 is then located, typically by the anatomical landmark of a second upper molar. (Greater palatine foramen 202 is typically located 1 cm medial to the second upper molar at the border between the hard and the soft palates.) A punch is used to puncture the mucosa covering foramen 202, providing access to a greater palatine canal 204 and foramen 202, at a puncture step 206.

As shown in FIG. 5A, forward tool 20 is advanced into greater palatine canal 204, at a forward preparation step 208. Distal tip 40 of forward tool 20 applies a forward longitudinal force in order to open a passage through the canal. Forward tool 20 is typically introduced up to about 2 cm past foramen 202, until distal tip 40 of the tool approaches a vicinity of a sphenopalatine ganglion (SPG) 210. Forward tool 20 is then completely withdrawn from the canal.

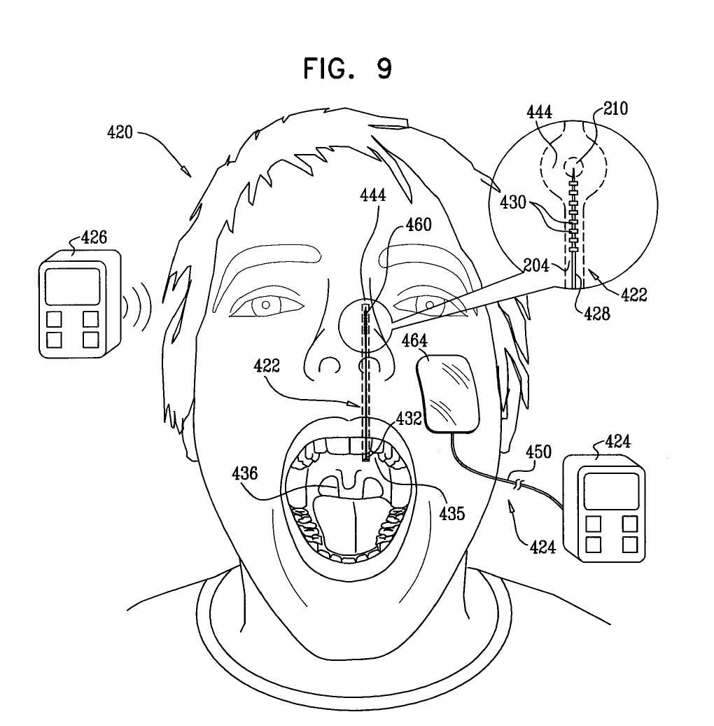

For some applications, during and/or after performing forward preparation step 208, a probe system is used for ascertaining whether a probe has been properly positioned within greater palatine canal 204, using techniques described hereinbelow with reference to FIGS. 9-19. Typically, once proper positioning has been ascertained, the probe is removed from the canal, and a neural stimulator is introduced into the bony canal in the same position and orientation the probe previously occupied. For some applications, after proper positioning has been ascertained, but before the neural stimulator has been introduced, additional preparation of the canal is performed using forward tool 20 and/or abrading tools 120.

As shown in FIG. 5B, a series of abrading tools 120, having successively greater diameters D₅, as described hereinabove with reference to FIG. 3, is typically used to widen the path through the canal created using forward tool 20, at an abrasion step 212. First, the narrowest abrading tool 120 of the series (e.g., having a diameter D₅ of 1.5 mm) is introduced through the path created by forward tool 20, keeping tight contact between the instrument and a posterior wall 220 of greater palatine canal 204. A gentle forward-and-backward abrading maneuver is typically used. Abrading tool 120 is typically inserted into the greater palatine canal to a depth of about 20 mm. Alternatively, the depth of the greater palatine canal is measured prior to or during the implantation procedure, in which case the tip is inserted to the measured depth. For example, techniques may be used that are described hereinbelow with reference to FIGS. 9-19.

The first, narrowest, abrading tool 120 is removed, and abrasion step 212 of the surgical procedure is repeated using abrading tools 120 having successively greater diameters D₅, until greater palatine canal 204 is widened, typically, to about 2 mm. For some applications, the series of abrading tools 120 includes exactly three tools, having diameters D₅ of 1.5 mm, 1.8 mm, and 2.0 mm, respectively.

Reference is made to FIG. 7, which is a schematic illustration of an introducer tool 250 and a neural stimulator 252, in accordance with an embodiment of the present invention. At a stimulator implantation step 222 of the method of FIG. 6, neural stimulator 252, or another neural stimulator, is implanted in greater palatine canal 204 after the canal has been prepared, as described hereinabove.

Neural stimulator 252 typically comprises an elongated support element 258, one or more electrodes 260 fixed to the support element in a vicinity of a distal end thereof, and circuitry 262 coupled to the support element in a vicinity of a proximal end thereof. The support element is typically electrically insulated along the length thereof. Neural stimulator 252 may incorporate apparatus and techniques described hereinbelow with reference to FIGS. 9-19, and/or in one or more of the patent applications incorporated by reference hereinbelow.

A distal end of introducer tool 250 comprises a coupling element 270, to which a proximal end of neural stimulator 252 is temporary coupled prior to performing the implantation procedure. For example, a cord may be coupled to the proximal end of the neural stimulator, pass through the introducer tool, and be temporarily coupled to the proximal end of the introducer tool (e.g., with a knot) such that the cord is tense and holds the neural stimulator tightly to coupling element 270 (cord not shown in FIG. 7). For example, the cord may comprise a suture, string, or wire.

For some applications, introducer tool 250 comprises a collar 272, which is configured to limit a depth of insertion of the introducer tool in the greater palatine canal. For example, the collar may be configured to limit the depth of insertion of the distal tip of the neural stimulator to the estimated distance from the bottom of the hard palate to the SPG in a typical patient, e.g., between about 23 and about 33 mm, e.g., about 28 mm. For some applications, the collar comprises a plastic tube placed around all or a portion of the shaft of the introducer tool. For some applications, the introducer tool is shaped so as to define a bend 276 slightly proximal to collar 272. The bend is ergonomically helpful to the healthcare worker performing the implantation procedure. For example, an angle θ (theta) between an axis of a portion of the tool distal to bend 276 and an axis of a portion of the tool proximal to the bend may be between about 10 and about 30 degrees, e.g., between about 15 and 17 degrees.

During the implantation procedure, neural stimulator 252 is passed through the mucosa lining the hard palate of the oral cavity and the greater palatine foramen, and into the passage opened in the greater palatine canal. When the introducer and neural stimulator have reached the desired location, such as using techniques described hereinbelow with reference to FIGS. 9-19 for detecting passage of the stimulator out of the distal end of the canal, the neural stimulator is decoupled from the introducer tool (such as by cutting the cord). The introducer tool is withdrawn, leaving the distal end of the neural stimulator implanted in the vicinity of the SPG, and at least a proximal portion of support element 258 implanted in the greater palatine canal.

For some applications, verification and/or optimization of the electrode nerve interface after the electrodes are placed is performed by observing the effects of stimulation on one or more physiological responses. Potential observations include, but are not limited to: (1) evaluating the vasodilatation of blood vessels of the eye, (2) assessment of cerebral blood flow (e.g., changes in blood flow) by using Doppler (e.g., transcranial Doppler or laser Doppler), (3) assessment of forehead perfusion by using Laser-Doppler, and (4) assessment of forehead perfusion by a temperature sensor. For some applications, one or more of the techniques for accurately placing the stimulator are used that are described hereinbelow with reference to FIGS. 9-19.

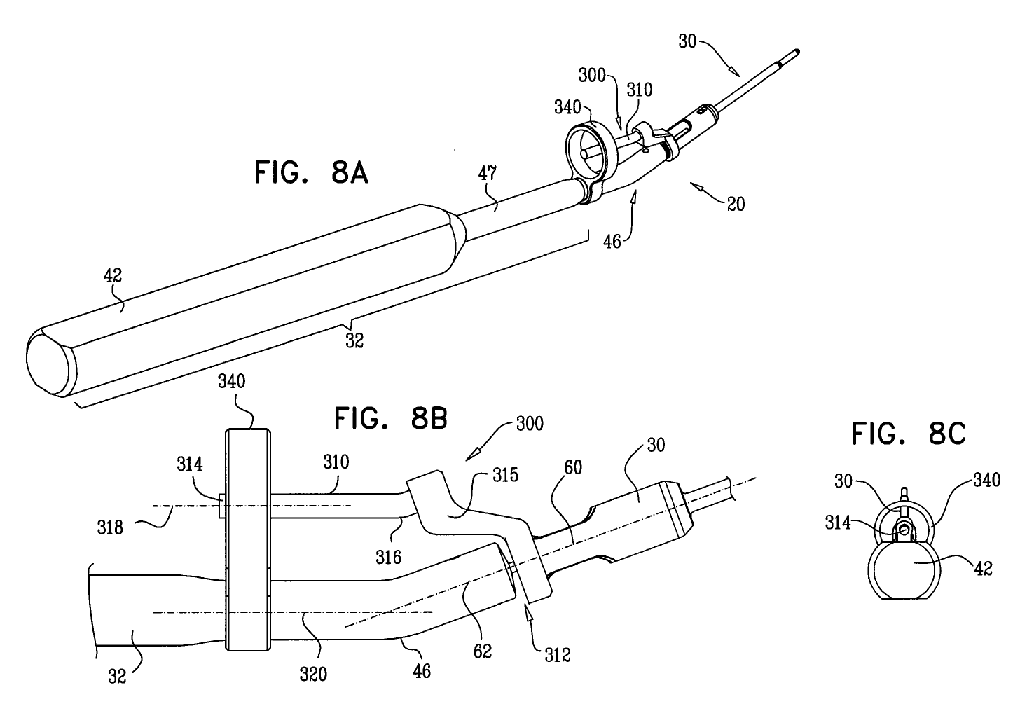

FIGS. 8A-C are schematic illustrations of a target sight 300 applied to forward tool 20, in accordance with an embodiment of the present invention. FIGS. 8A and 8B are two side views of the tool, and FIG. 8C is a view from the proximal end of handle 42. Sight 300 helps the surgeon aim the forward tool as the tool is advanced through the canal.

Sight 300 comprises an aiming element 310 that is coupled to distal rod 30, and extends in a generally proximal direction toward proximal shaft 32, past an articulation site 312 at which distal rod 30 articulates with proximal shaft 32. For some applications, aiming element 310 comprises a rod. Aiming element 310, and a proximal guide tip 314 thereof, move with respect to proximal shaft 32 as distal element 30 articulates with proximal shaft 32. Aiming element 310 thus indicates an alignment of distal rod 30 with respect to proximal shaft 32, e.g., a direction and/or extent of displacement from proper alignment of the distal rod with respect to the proximal shaft. Typically, aiming element 310 is coupled near a proximal end of the distal rod, e.g., within 20 mm of bend 46 of proximal shaft 32. The aiming element may be coupled directly to the distal rod, or indirectly by a coupling element 315.

For some applications, aiming element 310 is shaped so as to define a bend 316, which is oriented in the same direction as, and has the same angle as, bend 46 of proximal shaft 32. As a result, a central longitudinal axis 318 of the aiming element is parallel to a central longitudinal shaft axis 320 of proximal shaft 32 proximal to bend 46, when central longitudinal rod axis 60 of distal rod 30 is parallel to central longitudinal shaft axis 62 of proximal shaft 32 distal to bend 46.

For some applications, sight 300 further comprises an aiming ring 340, which is coupled to proximal shaft 32, typically proximal to bend 46. Aiming ring 340 and aiming element 310 are arranged such that proximal guide tip 314 of the aiming element can easily be observed with respect to the aiming ring.

Typically, as can be best seen in FIGS. 8B and 8C, proximal guide tip 314 is approximately centered with respect to the aiming ring when central longitudinal rod axis 60 of distal rod 30 is parallel to central longitudinal shaft axis 62 of proximal shaft 32 distal to bend 46, i.e., when the distal rod is properly aligned with respect to the proximal shaft. As the distal rod articulates in a first direction with respect to the proximal shaft, the proximal guide tip moves with respect to the aiming ring in a second direction generally opposite the first direction.

During an implantation procedure, as the surgeon advances distal rod 30 through the canal, he or she monitors the position of proximal guide tip 314 with respect to aiming ring 340. If the proximal guide tip becomes deflected from center in a given direction (i.e., the distal rod is not properly aligned with respect to the proximal shaft), the surgeon moves handle 42 and proximal shaft 32 in this given direction, thereby bringing the guide tip back to center. As a result, the proximal shaft becomes properly realigned with the distal rod and the direction of the canal, thereby reducing the risk of the distal rod penetrating the wall of the canal.

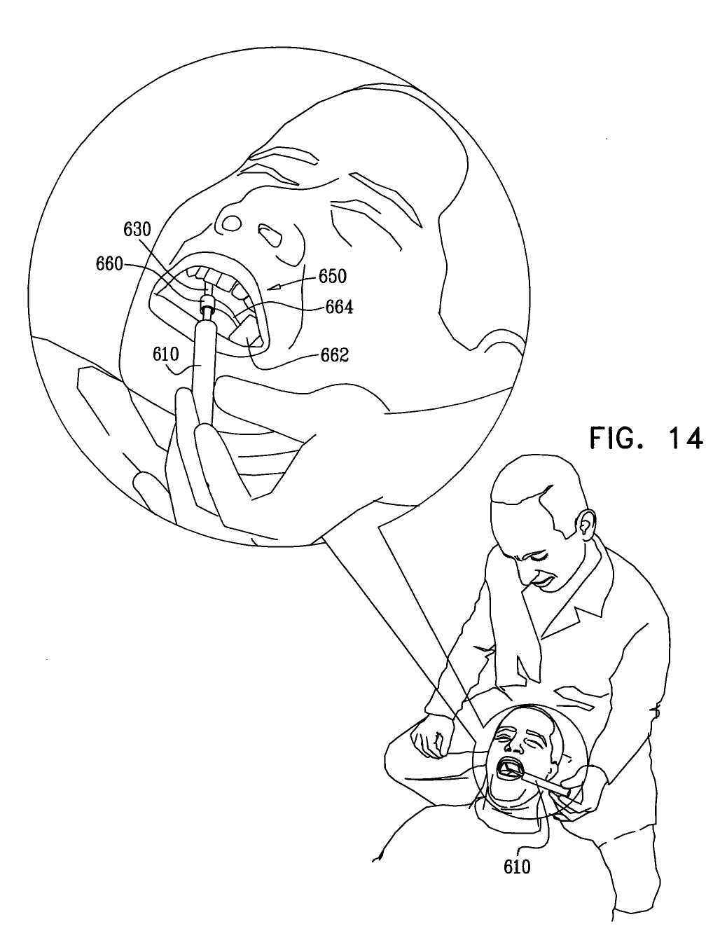

For some applications, the surgeon uses the correctly determined orientation of the proximal shaft for guiding additional tools into the canal in the same direction. Optionally, the surgeon uses a guidance jig, such as described hereinbelow with reference to FIG. 14, to guide and direct the additional tools along the same advancement route to the bony canal as taken by the proximal shaft. The additional tools may include, for example, introducer tool 250, described hereinabove with reference to FIG. 7.

In an embodiment of the present invention, abrading tool 120, described hereinabove with reference to FIGS. 3 and 4A-B, comprises target sight 300.

For some applications, techniques described herein are practiced in combination with techniques described in US Patent Application Publication 2006/0195169 to Gross et al., entitled, “Surgical tools and techniques for stimulation,” which is incorporated herein by reference.

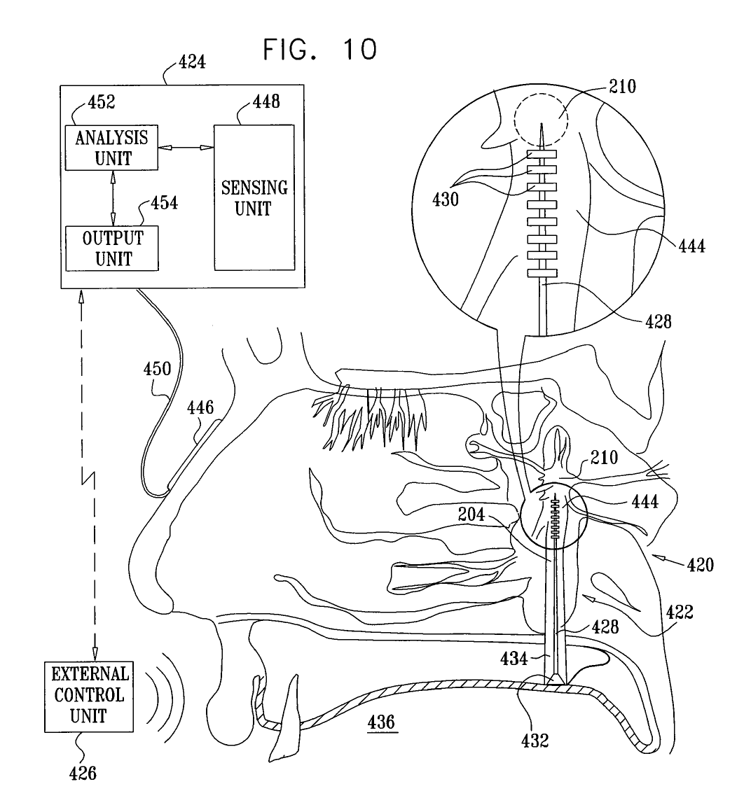

FIGS. 9 and 10 are schematic illustrations of a neural stimulation system 420 applied to a subject, shown in frontal view and cross-sectional view, respectively, in accordance with an embodiment of the present invention. System 420 comprises a neural stimulator 422, an external electrode positioning unit 424, and an external control unit 426. Stimulator 422 typically comprises an elongated support element 428, one or more electrodes 430 fixed to the support element in a vicinity of a distal end thereof, and circuitry 432 coupled to the support element in a vicinity of a proximal end thereof. The support element is typically electrically insulated along the length thereof. For some applications, a single unit serves as both external control unit 426 and external electrode positioning unit 424.

For some applications, neural stimulator 422 is configured to be implanted in the subject. For other applications, neural stimulator 422 is configured to be temporarily placed in the subject during a positioning phase of an implantation procedure, for ascertaining a desired implantation location. Once the implantation location has been ascertained, the neural stimulator is withdrawn from the subject, and an implantable neural stimulator is implanted at the ascertained implantation location. (It is noted that once the temporary neural stimulator has successfully passed through the canal, the implantable neural stimulator nearly always follows the same path through the canal when subsequently inserted.) Such a temporary neural stimulator may be used, for example, to enable the use of different electrical parameters (e.g., frequency) during the implantation procedure than during therapeutic stimulation after implantation. The different electrical parameters may enable clearer sensing of electrical parameters, such as impedance or voltage, as described hereinbelow. Although an implantable neural stimulator could be configured to enable programming of different parameters during the implantation procedure and therapeutic stimulation, elements necessary to enable such programmability may cause an undesired increase in the size, complexity, and/or power consumption of the implantable neural stimulator. Alternatively or additionally, such a temporary neural stimulator may be coupled to external electrode positioning unit 424 and/or external control unit 426 by wires, while the implantable neural stimulator is wireless, as described hereinbelow.

For some applications, the temporary neural stimulator is inserted using an introducer tool to which the temporary neural stimulator is temporarily coupled at the distal end of the tool. For example, the introducer tool may be similar in some respects to introducer tool 250 described hereinbelow with reference to FIG. 7. After identifying the appropriate implantation location, the healthcare worker withdraws the introducer tool, and decouples the temporary neural stimulator from the tool. The healthcare worker couples the implantable neural stimulator to the tool, and uses the tool to implant the implantable neural stimulator at the previously identified implantation location. For some applications, the healthcare worker notes the insertion depth of the introducer tool (such as by using insertion depth markings on the tool), and uses the tool to implant the implantable neural stimulator at the previously noted insertion depth, or at a slightly greater depth (e.g., between about 1 and about 2 mm deeper), such that the stimulator is positioned at the ascertained implantation location. Alternatively, guidance jig 650, described hereinbelow with reference to FIG. 14, may be used.

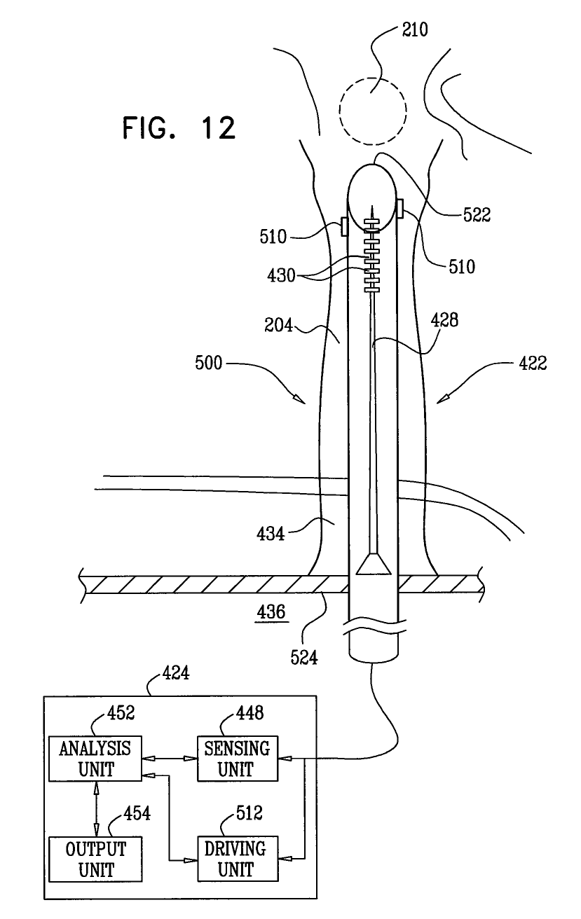

For other applications, the temporary neural stimulator is inserted using an introducer tool through which the temporary neural stimulator is passed. For example, the introducer tool may be similar in some respects to introducer tool 500 described hereinbelow with reference to FIG. 12. The introducer tool is left in place as the temporary neural stimulator is withdrawn, and thus serves to indicate the ascertained implantation location when the implantable neural stimulator is subsequently implanted using the introducer tool, by passing the implantable neural stimulator through the tool.

In an embodiment of the present invention, stimulator 422 is configured to be passed through a greater palatine foramen 434 of a hard palate 435 of an oral cavity 436 of the subject into greater palatine canal 204, such that electrodes 430 are brought into a vicinity of sphenopalatine ganglion (SPG) 210. For some applications, the entire stimulator is contained within greater palatine canal 204, while for other applications, at least a portion of the circuitry and/or the support element are positioned submucosally in the oral cavity. For clarity of illustration, the greater and lesser palatine nerves, and the greater and lesser palatine arteries are not shown in FIG. 10. During an implantation procedure, stimulator 422 is typically passed through greater palatine foramen 434 adjacent to the greater palatine nerve and artery.

For some applications, circuitry 432 comprises a wireless coupling element (which typically comprises a coil), and additional elements, such as one or more rectifiers, capacitors, amplifiers, or filters. One or more leads (not shown in FIG. 9), which pass along, through, or around support element 428, couple electrodes 430 to circuitry 432. Alternatively, the leads function as the support element, i.e., the support element does not comprise any structural elements in addition to the leads. Further alternatively, the leads provide a substantial portion of the structural support of the support element, and the balance of the structural support is provided by other elements. For example, support element 428 may comprise the leads and a flexible sleeve surrounding the leads; the leads supply most of the structural support of the support element, while the sleeve allows smooth passage of the leads through the greater palatine canal. For some applications, neural stimulation system 420 utilizes techniques described in US Patent Application Publication 2006/0287677 and/or other patent applications assigned to the assignee of the present application and incorporated by reference hereinbelow.

In an embodiment of the present invention, external electrode positioning unit 424 is configured to aid in the accurate positioning of electrodes 430 in a pterygopalatine fossa 444 in a vicinity of SPG 210. Neural stimulation system 420 comprises one or more external electrodes 446, which are configured to be placed at respective sensing sites on an external surface of a body of the subject. Typically, the external surface may be skin or hair of a head of the subject, such as skin of the face (including the ears) of the subject, or of the scalp of the subject. Sensing sites on the skin of the face of subject may include, for example, one or more of the cheeks, nose (e.g., the side of the nose), lips, or forehead of the subject. By way of example, FIG. 9 shows one of external electrodes 446 placed about in the center of the cheek, and FIG. 10 shows one of external electrodes 446 placed on the nose.

Typically, each of external electrodes 446 comprises a mechanical body-surface-coupling element, which is configured to temporarily couple the electrode to the skin or hair of the subject. For example, the coupling element may comprise an adhesive, such as provided on some ECG electrodes, or a suction element, such as provided on other ECG electrodes. Alternatively, the coupling element may comprise a hook, e.g., configured to couple the external electrode to a lip of the subject, as is known in the dental electrode art.

External electrode positioning unit 424 further comprises a sensing unit 448, an analysis unit 452, and an output unit 454, as shown schematically in FIG. 10. For some applications, the sensing unit, analysis unit, and output unit are contained within a separate housing, while for other applications, the sensing and analysis units are elements of external control unit 426. Sensing unit 448 is typically coupled to external electrodes 446 by one or more conductors 450, e.g., wires. The sensing unit is configured to generally constantly sense an electrical parameter at the sites using external electrodes 446 while electrodes 430 are electrically activated and advanced at least partially through the bony canal. For some applications, the sensed parameter is an electrical impedance, a voltage, or a current. For some applications, electrodes 430 are activated at a plurality of frequencies, e.g., by repeatedly cycling between the different frequencies, and the parameter is sensed at each of the frequencies. For some applications, external control unit 426 comprises a driving unit that electrically activates electrodes 430, while for other applications, external electrode positioning unit 424 comprises a driving unit that electrically activates electrodes 430.

In an embodiment of the present invention, analysis unit 452 is configured to detect passage of electrodes 430 out of a distal end of the bony canal responsively to a change in the sensed electrical parameter. Analysis unit 452 may detect the change using one or more of the following techniques:
Analysis unit 452 repeatedly compares the measured parameter with a threshold value, and detects the change when the measured parameter crosses the threshold value.
Analysis unit 452 ascertains a baseline value of the measured parameter, such as by averaging measured values over a first number of seconds of the implantation procedure. After ascertaining the baseline value, the analysis unit repeatedly calculates a percentage change of the currently measured value of the parameter compared to the baseline value. The analysis unit detects the change when the percentage change exceeds a threshold percentage value. Alternatively, the analysis unit detects the change when a difference between the current measured value of the parameter and the baseline value exceed a threshold value.
Analysis unit 452 repeatedly calculates a rate of change of the measured value of the parameter, and detects the change when the rate of change exceeds a threshold rate of change value.

For example, for applications in which the parameter is impedance, the analysis unit may detect the passage responsively to a change in the sensed impedance, such as a reduction or increase in the sensed impedance, e.g., of at least 1%, such as at least 3%, at least 5%, or at least 10%, or a change in a gradient of the impedance. Tissue outside of the bony canal, such as of the pterygopalatine fossa, generally has a measurably lower impedance than tissue of the bony canal, such as the greater palatine canal. The measured impedance thus falls as electrodes 430 pass from within the bony canal to outside the bony canal. For applications in which the parameter is voltage or current, the analysis unit may detect the passage responsively to a change in the parameter, such as an increase or reduction in the parameter, e.g., of at least 1%, such as at least 3%, at least 5%, or at least 10%, or a change in a gradient of the voltage or current.

In this embodiment, output unit 454 is configured to generate an output indicative of the detected passage. For example, the output may include an audible tone and/or a visible signal, and/or an electrical signal communicated to external control unit 426 and/or a separate monitoring device. Responsively to the output, the healthcare worker performing the electrode implantation procedure ascertains that electrodes 430 are positioned at the desired implantation location. For some applications, the healthcare worker implants the electrodes at the implantation location, or a certain distance further into the fossa, e.g., between about 1 and about 2 mm. For other applications, the electrodes are temporary electrodes, and the healthcare worker withdraws the temporary electrodes from the subject after ascertaining the implantation location, and implants other implantable electrodes at the ascertained implantation location, or a certain distance further into the fossa, e.g., between about 1 and about 2 mm.

In another embodiment of the present invention, output unit 454 of external electrode positioning unit 424 generally continuously generates an output indicative of a value of the sensed electrical parameter. The healthcare worker performing the procedure detects passage of electrodes 430 out of the distal end of the bony canal responsively to a change in the sensed electrical parameter, as detected in the output by the healthcare worker. For example, output unit 454 may include a display that displays a numerical value of the sensed parameter, and/or an audio generator that generates a tone having a frequency indicative of a magnitude of the sensed parameter. In this embodiment, external electrode positioning unit 424 typically does not automatically detect the change in the sensed parameter, and thus does not necessarily comprise analysis unit 452. Alternatively, external electrode positioning unit 424 implements both techniques in combination, i.e., (a) detects the change in the sensed parameter and generates an output responsively thereto, and (b) outputs a signal indicative of the measured value of the sensed parameter for consideration by the healthcare worker.

For some applications, external electrode positioning unit 424 is coupled to external control unit 426, and drives the external control unit to electrically activate electrodes 430 during the electrode placement procedure. Alternatively, for other applications, the electrode positioning unit is not operatively coupled to the external control unit, and a healthcare worker triggers the external control unit to electrically activate electrodes 430 during the electrode placement procedure.

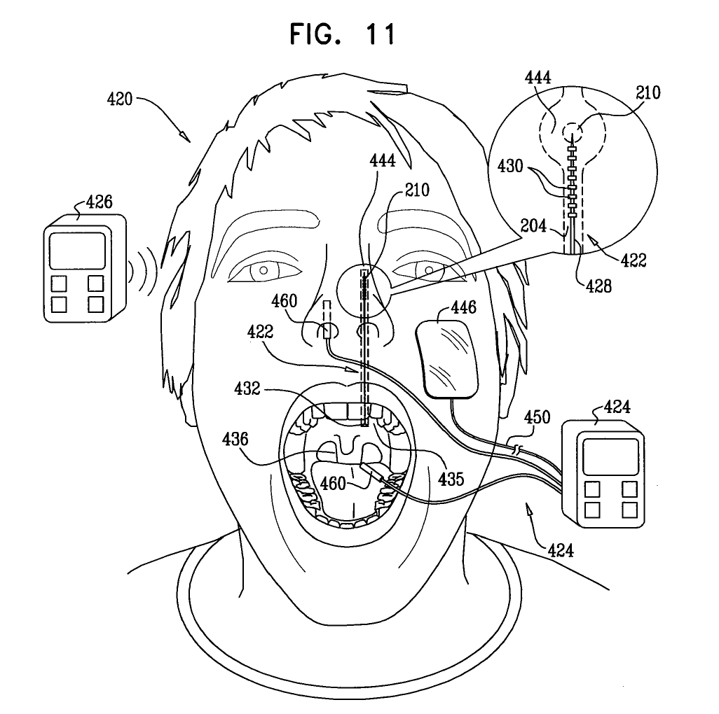

In an embodiment of the present invention, external electrodes 446 are not directly or indirectly coupled to electrodes 430 by any metal conductors (e.g., wires, leads or circuitry). External control unit 426 is configured to transmit energy to neural stimulator 422 either wirelessly or over one or more leads, in order to electrically activate electrodes 430, by driving a current between at least two of the electrodes. Such activation causes the electrodes to generate an electric field, which is sensed by external electrodes 446. For applications in which external control unit 426 wirelessly transmits the energy to stimulator 422, an external wireless coupling element, coupled to the external control unit, wirelessly transmits energy to a wireless coupling element of circuitry 432 of stimulator 422. Each of the wireless coupling elements typically comprises at least one coil. For some applications, the wireless coupling elements are wirelessly coupled to one another using induction, such as when the wireless coupling elements are positioned in close proximity to one another. Alternatively, the wireless coupling elements are wirelessly coupled to one another using RF energy, such as when the wireless coupling elements are positioned at a greater distance from each other. Further alternatively, the wireless coupling elements are wirelessly coupled to one another using another form of energy, such as ultrasound energy, in which case the wireless coupling elements comprises ultrasound transducers, e.g., piezoelectric transducers. For some applications, the wireless transmission of energy and/or data is performed using techniques described in US Patent Application Publication 2006/0287677, such as with reference to FIGS. 11A-B, 12, 13, 14A-B, and/or 15 thereof.

In another embodiment of the present invention, external electrodes 446 are coupled to electrodes 430 by at least one metal conductor, e.g., wire, typically indirectly via one or more leads and circuitry of external electrode positioning unit 424 and/or external control unit 426. External electrode positioning unit 424 and/or external control unit 426 drives a current between at least one of external electrode 446 and at least one of electrodes 430, and sensing unit 448 electrodes senses an electrical parameter of the current. This approach generally produces a stronger signal with a greater signal-to-noise ratio. For example, circuitry of external electrode positioning unit 424 may be coupled to external control unit 426, which is coupled to circuitry 432 of stimulator 422 by at least one conductor, e.g., wire. Alternatively, the circuitry of external electrode positioning unit 424 may be coupled directly to the circuitry 432 of stimulator 422 by at least one conductor, e.g., wire, rather than via external control unit 426. For some applications, stimulator 422 is configured such that during the electrode placement procedure, electrodes 430 are activated using energy provided to stimulator 422 via one or more metal conductors, e.g., wires, and during therapeutic use of the stimulator after electrode placement, electrodes 430 are activated using energy wirelessly transmitted to stimulator 422. For example, the use of such wired coupling during the electrode placement procedure may enable the use of different electrical parameters during the electrode placement procedure than during subsequent therapeutic use of the stimulator.

For some applications, electrodes 430 are temporary electrodes, and the healthcare worker withdraws the temporary electrodes from the subject after ascertaining the implantation location, and implants other implantable electrodes at the ascertained implantation location.

For some applications, support element 428 has a length of between about 1.8 and about 4 cm, such as between about 2.6 cm and about 3 cm, e.g., about 2.8 cm, and has a curvature that follows that of the greater palatine canal. For some applications, support element 428 has a diameter at its widest portion of between about 1 and about 4 mm. For some applications, support element 428 comprises a tube. For some applications, support element 428 is semi-rigid (i.e., it generally keeps its original shape during a placement procedure). For example, support element 428 may be sufficiently rigid to enable insertion of the support element into a body of the subject by pushing from a vicinity of a proximal end of the support element. For some applications, support element 428 and electrodes 430 together are similar to conventional concentric needle electrodes, such as Medtronic, Inc. needle electrode model DCN50, or Oxford Instruments Plc. needle electrode models X53153, X53155, X53156, X53158, or X53159.

Each of electrodes 430 typically comprises a suitable conductive material, for example, a physiologically-acceptable material such as silver, iridium, platinum, a platinum iridium alloy, titanium, nitinol, or a nickel-chrome alloy. Electrodes 430 are insulated from one another with a physiologically-acceptable material such as polyethylene, polyurethane, or a co-polymer of either of these. For some applications, the electrodes are spiral in shape, for better contact, and may have a hook shaped distal end for hooking into or near the SPG. Alternatively or additionally, the electrodes may comprise simple wire electrodes, spring-loaded “crocodile” electrodes, or adhesive probes, as appropriate. For some applications, the electrodes are coated with a biocompatible material configured to enhance the surface area of the electrodes, thereby increasing the capacitance and reducing the resistance of the electrodes. For example, the material may comprise a platinum/iridium alloy, and/or may be applied with a sputtering process, such as commercially available from Johnson Matthey Plc, Advanced Metals Technology division (London, UK).

In an embodiment of the present invention, external electrode positioning unit 424 is electrically coupled to two or more of electrodes 430 of stimulator 422. External control unit 426 or external electrode positioning unit 424 activate the electrodes by driving a current between the electrodes, and sensing unit 448 of positioning unit 424 is configured to generally constantly sense an electrical parameter of the current while the electrodes are advanced at least partially through the bony canal. For some applications, the sensed parameter is an electrical impedance, a voltage, or a current. A change in the sensed parameter indicates passage of the electrodes out of the canal, such as into pterygopalatine fossa 444 in a vicinity of SPG 210. In this embodiment, neural stimulation system 420 does not necessarily comprise external electrodes 446. For some applications, electrodes 430 are activated at a plurality of frequencies, e.g., by repeatedly cycling between the different frequencies, and the parameter is sensed at each of the frequencies.

For some applications, a single unit serves as both external control unit 426 and external electrode positioning unit 424. Alternatively, one or more of sensing unit 448, analysis unit 452, and output unit 454 of external electrode positioning unit 424 are contained within the housing, and the external electrode positioning unit is coupled to external control unit 426, which may drive the current between the two or more electrodes 430.

For some applications, as in some of the embodiments described above, analysis unit 452 is configured to detect passage of electrodes 430 out of a distal end of the bony canal responsively to a change in the sensed electrical parameter. For some applications, analysis unit 452 detects the change using one or more of the techniques for doing so described hereinabove.

For example, for applications in which the parameter is impedance, the analysis unit may detect the passage responsively to a change in the sensed impedance, such as a reduction or increase in the sensed impedance, e.g., of at least 1%, such as at least 3%, at least 5%, or at least 10%, or a change in a gradient of the impedance. Tissue outside of the bony canal, such as of the pterygopalatine fossa, generally has a measurably lower impedance than tissue of the bony canal, such as the greater palatine canal. The measured impedance thus falls as electrodes 430 pass from within the bony canal to outside the bony canal. For applications in which the parameter is voltage or current, the analysis unit may detect the passage responsively to a change in the parameter, such as an increase or reduction, e.g., of at least 1%, such as at least 3%, at least 5%, or at least 10%, or a change in a gradient of the voltage or current.

In this embodiment, output unit 454 is configured to generate an output indicative of the detected passage. For example, the output may include an audible tone and/or a visible signal, and/or an electrical signal communicated to external control unit 426 and/or a separate monitoring device. Responsively to the output, the healthcare worker performing the electrode implantation procedure ascertains that electrodes 430 are positioned at the desired implantation location. For some applications, the healthcare worker implants the electrodes at the implantation location, or a certain distance further into the fossa, e.g., between about 1 and about 2 mm. For other applications, the electrode are temporary electrodes, and the healthcare worker withdraws the temporary electrodes from the subject after ascertaining the implantation location, and implants implantable electrodes at the ascertained implantation location, or a certain distance further into the fossa, e.g., between about 1 and about 2 mm.

Alternatively, as in some of the embodiments described above, output unit 454 of external electrode positioning unit 424 generally continuously generates an output indicative of a value of the sensed electrical parameter. The healthcare worker performing the procedure detects passage of electrodes 430 out of the distal end of the bony canal responsively to a change in the sensed electrical parameter, as detected in the output by the healthcare worker. For example, output unit 454 may include a display that displays a numerical value of the sensed parameter, and/or an audio generator that generates a tone having a frequency indicative of a magnitude of the sensed parameter. In this embodiment, external electrode positioning unit 424 typically does not automatically detect the change in the sensed parameter, and thus does not necessarily comprise analysis unit 452. Alternatively, external electrode positioning unit 424 implements both techniques in combination, i.e., (a) detects the change in the electrical parameter and generates an output responsively thereto, and (b) outputs a signal indicative of the measured value of the sensed parameter for consideration by the healthcare worker.

Reference is made to FIG. 11, which is a schematic illustration of an alternative configuration of neural stimulation system 420, in accordance with an embodiment of the present invention. In this embodiment, in addition to or instead of external electrodes 446, system 420 comprises one or more intracavitary sensing electrodes 460, which are configured to be temporarily placed in a cavity of the subject during a procedure for placing electrodes 430, and are used instead of or in addition to external electrodes 446. For example, the cavity may be a facial cavity, e.g., oral cavity 436 or a nasal cavity of the subject.

Reference is made to FIG. 12, which is a schematic illustration of an introducer tool 500 for implanting neural stimulator 422, in accordance with an embodiment of the present invention. For some applications, introducer tool 500 comprises one or more introducer electrodes 510 coupled thereto, typically in a vicinity of a distal end thereof. External electrode positioning unit 424 is electrically coupled to two of introducer electrodes 510. Sensing unit 448 is configured to generally constantly sense an electrical parameter while the two or more introducer electrodes 510 are electrically activated (by driving a current between the two or more electrodes) and advanced at least partially through the bony canal during a procedure for implanting neural stimulator 422. For some applications, the sensed parameter is an electrical impedance, a voltage, or a current. A change in the sensed parameter indicates passage of the introducer electrodes out of the canal, such as into pterygopalatine fossa 444 in a vicinity of SPG 210. For some applications, electrodes 510 are activated at a plurality of frequencies, e.g., by repeatedly cycling between the different frequencies, and the parameter is sensed at each of the frequencies. In this embodiment, neural stimulation system 420 does not necessarily comprise external electrodes 446 or intracavitary electrodes 460. For some applications, external electrode positioning unit 424 comprises a driving unit 512, which drives the current between the two or more introducer electrodes 510, while for other application, a driving unit of external control unit 426 drives the current between the two or more introducer electrodes.

For some applications, as in some of the embodiments described above, analysis unit 452 is configured to detect passage of electrodes 430 out of a distal end of the bony canal responsively to a change in the sensed electrical parameter, such as described hereinabove regarding the embodiment in which the electrical parameter is sensed while two or more of electrodes 430 are electrically activated. For some applications, analysis unit 452 detects the change using one or more of the techniques for doing so described hereinabove. In this embodiment, output unit 454 is configured to generate an output indicative of the detected passage, as described hereinabove. Alternatively, as in some of the embodiments described above, output unit 454 of external electrode positioning unit 424 generally continuously generates an output indicative of a value of the sensed electrical parameter, as described hereinabove. Further alternatively, external electrode positioning unit 424 implements both techniques in combination.

For some applications, introducer tool 500 comprises a rigid tube which is shaped so as to define a sharp distal tip 522. Prior to or during an implantation procedure, neural stimulator 422 is placed in the bore of the tube. The tube is passed through mucosa 524 lining the hard palate of oral cavity 436 and greater palatine foramen 434, into greater palatine canal 204. For applications in which the introducer tool comprises introducer electrodes 510, external electrode positioning unit 24 senses when introducer electrodes 510 have passed from greater palatine canal 204, as described above. When the introducer and neural stimulator have reached the desired location, the tube is withdrawn, leaving the distal end of the neural stimulator implanted in the vicinity of SPG 210, and at least a proximal portion of support element 428 implanted in greater palatine canal 204. Alternatively, the tube is first passed into canal 204, and stimulator 422 is subsequently introduced into the bore of the tube. The tube is typically passed through mucosa 524 without requiring a prior surgical incision in the mucosa, i.e., without requiring the use of a surgical knife. For some applications, introducer tool 500 is used to delivery neural stimulator 422 through a bony canal other than greater palatine canal 204.

In an embodiment of the present invention, external electrode positioning unit 424 is electrically coupled to at least one of electrodes 430 and at least one of introducer electrodes 510. Sensing unit 448 is configured to generally constantly sense an electrical parameter while the electrode and the introducer electrode are electrically activated (by driving a current between the electrode and the introducer electrode).

Reference is again made to FIG. 7, which is a schematic illustration of an introducer tool 250 for implanting neural stimulator 422, in accordance with an embodiment of the present invention. A distal end of introducer tool 250 comprises a coupling element 270, to which a proximal end of neural stimulator 422 is temporary coupled prior to performing the implantation procedure. For example, a cord may be coupled to the proximal end of the neural stimulator, pass through the introducer tool, and be temporarily coupled to the proximal end of the introducer tool (e.g., with a knot) such that the cord is tense and holds the neural stimulator tightly to coupling element 270 (cord not shown in FIG. 7). For example, the cord may comprise a suture, string, or wire.

For some applications, introducer tool 250 comprises a collar 272, which is configured to limit a depth of insertion of the introducer tool in the greater palatine canal. For example, the collar may be configured to limit the depth of insertion of the distal tip of the neural stimulator to the estimated distance from the bottom of the hard palate to the SPG in a typical patient, e.g., between about 23 and about 33 mm, e.g., about 28 mm. For some applications, the collar comprises a plastic tube placed around all or a portion of the shaft of the introducer tool. For some applications, the introducer tool is shaped so as to define a bend 276 slightly proximal to collar 272. The bend is ergonomically helpful to the healthcare worker performing the implantation procedure. For example, an angle θ (theta) between an axis of a portion of the tool distal to bend 276 and an axis of a portion of the tool proximal to the bend may be between about 10 and about 30 degrees, e.g., between about 15 and 17 degrees.

During the implantation procedure, neural stimulator 422 is passed through the mucosa lining the hard palate of the oral cavity and the greater palatine foramen, and into the greater palatine canal. When the introducer and neural stimulator have reached the desired location, such as using techniques herein for detecting passage of the stimulator out of the distal end of the canal, the neural stimulator is decoupled from the introducer tool (such as by cutting the cord). The introducer tool is withdrawn, leaving the distal end of the neural stimulator implanted in the vicinity of the SPG, and at least a proximal portion of support element 428 implanted in the greater palatine canal. The neural stimulator is typically passed through the mucosa without requiring a prior surgical incision in the mucosa, i.e., without requiring the use of a surgical knife. For some applications, the distal end of the neural stimulator is shaped so as to define a sharp punch to enable passage through the mucosa. For some applications, introducer tool 250 is used to delivery neural stimulator 422 through a bony canal other than the greater palatine canal.

In an embodiment of the present invention, during an implantation procedure, a distance that neural stimulator 422 has been advanced through the bony canal is mechanically measured (e.g., an advancement distance of a location on the neural stimulator at which one of electrodes 430 is positioned, or a distal tip of the stimulator). For example, an introducer tool, e.g., similar in some respects to introducer tool 500 or introducer tool 250, described hereinabove with reference to FIG. 12 or 7, respectively, may be used to insert the neural stimulator, and the introducer tool, or a slider attached thereto, may have marks thereon that indicate a depth of insertion. The distance may be measured manually by the healthcare worker performing the implantation procedure, or automatically, such as by external electrode positioning unit 424.

In an embodiment of the present invention, accurate placement of neural stimulator 422 is accomplished using a combination of two or more of the following techniques:
using one or more external electrodes 446 or intracavitary electrodes 460 to measure an electrical parameter while one or more electrodes 430 are activated, as described hereinabove with reference to FIGS. 9, 10, and 11;
using two or more of electrodes 430 and/or introducer electrodes 510 to measure an electrical parameter while the two or more electrodes are activated by driving a current therebetween, as described hereinabove with reference to FIG. 12;
mechanically measuring a distance that the distal tip of neural stimulator 422 has been advanced, as described hereinabove; and/or
observing or measuring at least one physiological indicator of cerebral blood flow (CBF) concurrently with or after placement of neural stimulator 422, as described hereinbelow.

In an embodiment of the present invention, the distance that the distal tip of neural stimulator advances is mechanically measured, such as described hereinabove. The measured distance is compared to a length of the bony canal, as estimated for subjects having typical anatomy. For example, the greater palatine canal typically has a length of between about 10 mm and about 22 mm. Successful passage of electrodes 430 out of the distal end of the canal is detected based on the measured electrical parameter only if the measured distance exceeds a threshold value based on the estimated length of the canal (e.g., the threshold value may be slightly less than the estimated length of the canal, or equal to the estimated length of the canal). Use of this technique generally reduces false positive detections of successful passage from the distal end of the canal. Detection of the passage of the electrodes out of the canal before the electrodes have been sufficiently advanced, as measured mechanically, indicates that the electrodes may have punctured through the wall of the canal. For some applications, the healthcare worker detects the passage based on both the measured electrical parameter and measured length of the canal. For other applications, such as when the distance is measured automatically, external electrode positioning unit 424 performs the detection based on both measurements. Further alternatively, such as when the distance is measured manually, the external electrode positioning unit generates an output indicative of passage out of the distal end of the canal based on the measured electrical parameter, and the healthcare worker interprets the output as being accurate only if the measured distance exceeds the threshold value.

In an embodiment of the present invention, system 420 comprises circuitry described in one or more of the patent applications incorporated herein by reference hereinbelow.

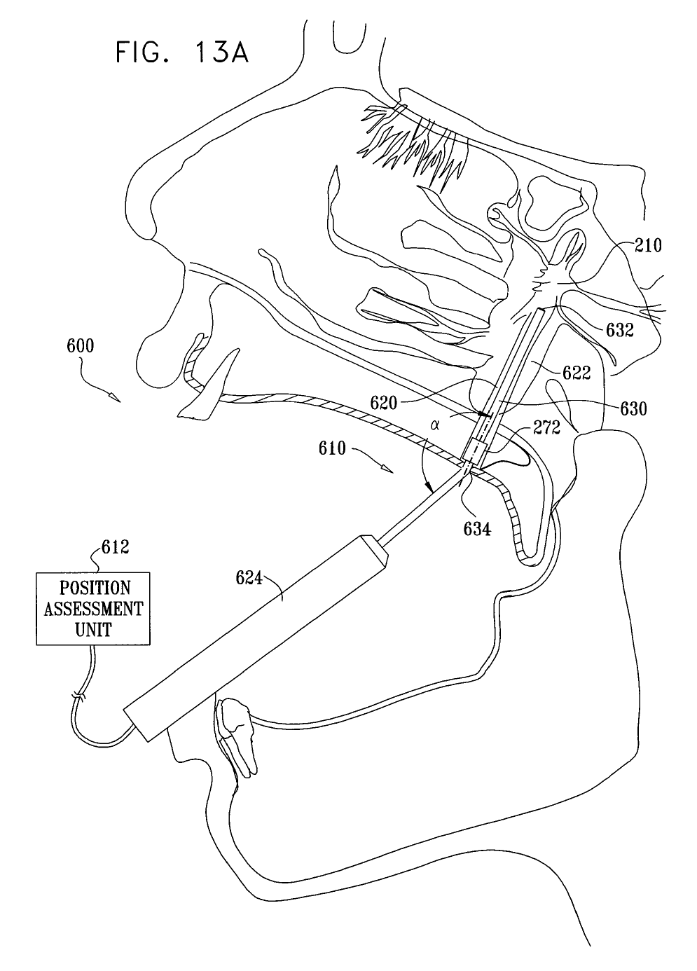

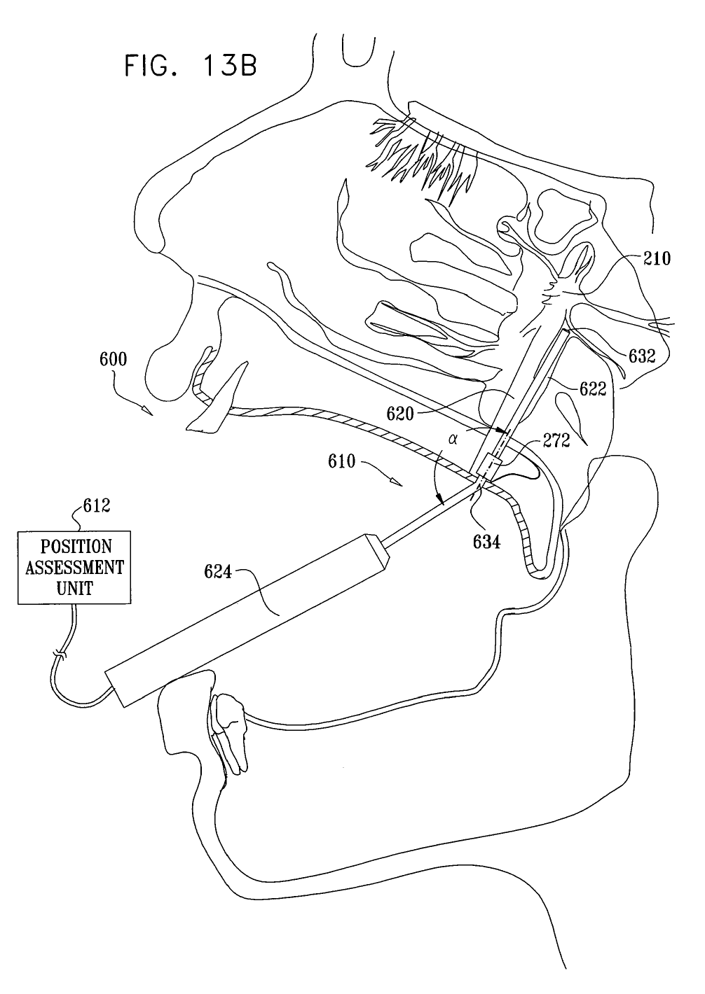

Reference is now made to FIGS. 13A and 13B, which are schematic illustrations of a probe system 600 applied to a subject, shown in cross-sectional view, in accordance with an embodiment of the present invention. Probe system 600 comprises a probe 610 and a position assessment unit 612. Probe system 600 aids in ascertaining whether probe 610 has been properly positioned in a bony canal, such as a greater palatine canal 620, as shown in FIG. 13A, rather than improperly positioned in adjacent tissue 622 outside canal 620, as shown in FIG. 13B.

Probe 610 typically comprises a handle 624 coupled to a shaft 630 that is configured to be partially introduced into the bony canal. The shaft may be solid or hollow (i.e., the shaft may comprise a tube). For some applications, at least a portion of position assessment unit 612 (e.g., all of the unit) is contained within handle 624. Probe 610 further comprises a position assessment element 632, which is typically positioned within 10 mm of a distal end of shaft 630. The portion of the shaft that is introduced into the bony canal typically has a greatest diameter of less than 1.5 mm, e.g., less than 1.2 mm, which allows the shaft to be introduced into a narrow bony canal, such a greater palatine canal. The shaft is sufficiently rigid to be pushed into the canal from the shaft's proximal end at the handle. For example, the shaft may be rigid or semi-rigid. (“Semi-rigid,” as used herein regarding the shaft, including in the claims, means sufficiently rigid to transfer forces in a longitudinal direction.) For some applications, the shaft has a length of between about 20 and about 40 mm, such as about 30 mm.

For some applications, shaft 630 is shaped so as to define a bend 634, which is ergonomically helpful to the healthcare worker performing the implantation procedure. For some applications, bend 634 has an angle α (alpha) of between about 150 and about 170 degrees, e.g., between about 163 and 165 degrees, e.g., 164 degrees. Alternatively, the bend is defined between the shaft and the handle. For some applications, shaft 630 comprises a collar 272, which is configured to limit a depth of insertion of the shaft in bony canal 620, such as the greater palatine canal, such as described hereinabove regarding introducer tool 250 of FIG. 7. For example, the collar may have a diameter of 3 mm. For some applications, handle 624 has a length of about 12 cm and a diameter of between about 1 and about 2 cm.

In some embodiments of the present invention, probe 610 is used during a surgical procedure for implanting a neural stimulator in the bony canal. Typically, local anesthetic is applied to the oral palatine mucosa and a greater palatine block is performed prior to a mucoperiosteal incision proximate the greater palatine foramen to reveal the contents of the foramen. Typically, the canal is widened using a series of one or more dilator tools, having successively greater distal shaft diameters. For some applications, the canal is widened using surgical methods and one or more of the dilator tools described hereinabove with reference to FIGS. 1-8C.

The healthcare worker performing the implantation procedure attempts to introduce shaft 630 with position assessment element 612 into the bony canal and advance the shaft through up to about 20 mm of the canal. However, because the healthcare worker does not always succeed, the shaft and position assessment element sometimes inadvertently never enter the proximal end of the canal, or, when the shaft and position assessment element do initially enter the proximal end of the canal, they accidentally puncture through the wall of the canal as they are advanced through the canal. In order to ensure successful positioning and advancing of the shaft within the canal, the healthcare worker uses probe system 600 to ascertain whether the position assessment element is within or outside the bony canal. For some applications, while advancing the shaft through the canal, the healthcare worker uses probe system 600 to confirm that the shaft is within the canal between one and five times as the shaft is advanced through the canal, or more than five times. Alternatively, while advancing the shaft through the canal, the healthcare worker generally constantly uses the probe system to confirm that the shaft is within the canal.

Reference is made to FIG. 14, which is a schematic illustration of a guidance jig 650, in accordance with an embodiment of the present invention. Typically, once proper positioning of probe 610 in the bony canal has been ascertained, the probe is removed from the canal, and a neural stimulator is introduced into the bony canal in the same position the probe previously occupied. For some applications, the neural stimulator is introduced using an introducer tool, which is introduced into the bony canal in the same position that probe previously occupied. For example, the neural stimulator may comprise neural stimulator 422, and/or the introducer tool may comprise introducer tool 250, both of which are described hereinabove with reference to FIG. 7, and may be implanted using techniques described herein with reference to FIG. 7. Guidance jig 650 guides and directs the neural stimulator along the same advancement route to the bony canal as taken by probe 610, and thus helps successfully position the neural stimulator in the bony canal in the same position the probe previously occupied in the canal.

In an embodiment of the present invention, guidance jig 650 comprises a guide element 660 that is adjustably coupled to a mouth prop 662, such as by an adjustably positionable arm 664. Guide element 660 guides the neural stimulator in the same orientation as probe 610. For some applications, the guide element comprises a tube through which pass shaft 630 of probe 610 and, subsequently, the neural stimulator and/or a neural stimulator introducer tool, such as introducer tool 250, described hereinabove with reference to FIG. 7. Mouth prop 662 is held firmly in place in the mouth, such as in the vestibule between the teeth and the cheek. Before and/or during introduction of probe 610 into the bony canal, the positioning of arm 664 is adjusted to hold guide element 660 in the desired orientation.

In an embodiment of the present invention, guidance jig 650 is not provided. Instead, the healthcare worker performing the implantation procedure manually inserts the neural stimulator along the same route used to successfully insert the probe into the bony canal. It is noted that once the probe has been successfully introduced into the body canal, the neural stimulator nearly always follows the same path through the canal when subsequently inserted.

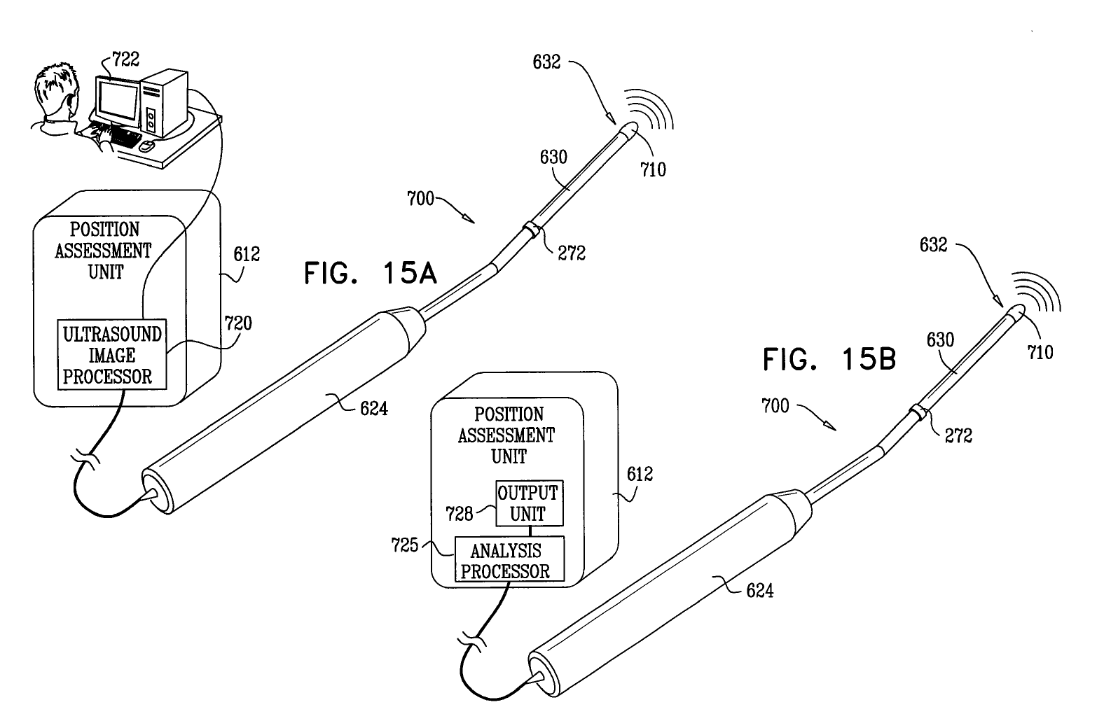

Reference is made to FIGS. 15A-B, which are schematic illustrations an ultrasound probe 700, in accordance with respective embodiments of the present invention. In these embodiments, position assessment element 632 comprises at least one ultrasound transducer 710 fixed to shaft 630, typically within 10 mm of a distal end thereof. Position assessment unit 612 analyzes the energy emitted by the at least one ultrasound transducer, and responsively to the analysis, ascertains whether the transducer is within or outside the bony canal.

For some applications, position assessment unit 612 analyzes the emitted ultrasonic energy to find a density of tissue within a certain distance of the at least one transducer (such as within between 0.5 and 5 mm of the at least one transducer, e.g., within 0.5 and 4 mm, such as within 3 mm). Responsively to the density of the tissue within the distance, the position assessment unit ascertains whether the transducer is within or outside the bony canal. If the probe is properly positioned with the bony canal, the at least one transducer will detect the bony wall of the canal within the distance. On the other hand, if the probe penetrates the wall of the canal into soft tissue surrounding the canal, the at least one transducer will detect only soft tissue, and not bone, within the distance. The position assessment unit recognizes that such soft tissue detected within the distance surrounds the canal, and that the probe has thus penetrated through the canal. For some applications, the at least one ultrasound transducer produces ultrasound energy at a frequency of between 0.5 and 60 MHz, such as between 5 and 15 MHz.

It is noted that conventional intravascular ultrasound systems typically detect and identify a continuous range of tissue densities, in order to differentiate between several tissue types and other materials such as plaque. In contrast, ultrasound probe 700 is typically used to distinguish between only two types of tissue (soft tissue and bone), or at most a few types of tissue (e.g., between two and five), and, optionally, undefined tissue having a density between the expected densities of soft tissue and bone.

In the configuration shown in FIG. 15A, position assessment unit 612 comprises an ultrasound image processor 720 and an imaging display unit 722. Image processor 720 processes the ultrasonic energy received from ultrasound transducer 710 to produce an ultrasound image of the area surrounding the transducer, and displays the image on imaging display unit 722. The healthcare worker analyzes the images to ascertain whether the transducer and shaft of the probe are properly positioned within the bony canal, rather than in surrounding soft tissue. Alternatively, image processor 720 analyzes the images to find a density of tissue near the transducer. In this case, position assessment unit 612 does not necessarily comprise imaging display unit 722.

In the configuration shown in FIG. 15B, position assessment unit 612 comprises an analysis processor 726 and an output unit 728. For some applications, output unit 728 is contained within handle 624 (configuration not shown), while for other application, the output unit is provided in a separate housing. Analysis processor 726 analyzes the ultrasonic energy produced by ultrasound transducer 710 to ascertain whether the transducer is adjacent to bone or soft tissue, i.e., whether the transducer is within the bony canal or in surrounding soft tissue. Output unit 728 is configured to generate an output indicative of the result of the analysis. For example, the output may include an audible tone and/or a visible signal, and/or an electrical signal communicated to a separate monitoring device. Alternatively, the position assessment unit is configured to generate an output indicative of a value of the density of the tissue near the transducer, and the healthcare worker performing the implantation procedure ascertains whether the transducer and shaft are within the bony canal responsively to the outputted value. For example, output unit 728 may include a display that displays a numerical value of the tissue density, and/or an audio generator that generates a tone having a frequency indicative of a magnitude of the tissue density.

Reference is made to FIG. 16, which is a schematic illustration a light-emitting probe 750, in accordance with an embodiment of the present invention. In this embodiment, position assessment element 632 comprises a light-emitting element 760 fixed to shaft 630, typically within 10 mm of a distal end thereof. Shaft 630 is advanced into the greater palatine canal. The emitted light is detected in the mouth or nose, or on an external surface of the face (e.g., the cheek), either visually by the healthcare worker performing the procedure, or using a light sensor, as described hereinbelow. The intensity of the light detected in the mouth or nose, or on the external surface of the face is less if light-emitting element 760 is properly positioned within the bony canal than if shaft 630 penetrates the wall of the canal into soft tissue surrounding the canal.

For some applications, light-emitting element 760 comprises an LED or other light source, which is typically positioned within 10 mm of a distal end of the shaft. Alternatively, light-emitting probe 750 comprises a light source 762 that is positioned remotely from light-emitting element 760, and transmits the generated light to the light-emitting element via one or more fiber-optic cables. For example, light source 762 may be positioned within handle 624 or position assessment unit 612.

For some applications, light-emitting element 760 is positioned generally at the distal tip of the probe 750, as shown in FIG. 16, such that the element emits light generally in all directions. For other applications, the light-emitting element is positioned on the side of the probe, such that the element emits light more strongly in the lateral direction of the element (configuration not shown). For these applications, emission of the light enables the healthcare worker to monitor the depth and rotation of the probe within the canal.

The generated light may be in the visible spectrum, e.g., white light or red light (between 350 and 750 nm, such as 620 nm), or may be in the non-visible spectrum, e.g., infrared. For example, the light may have a wavelength of between 350 and 1000 nm. For some applications, the light-emitting element is configured to emit the light intermittently, e.g., to blink, which generally enhances detection of the light because of the contrast between when the light is generated and not generated. Such intermittent light generation also reduces any heat generated by the light source. For example, the light may be intermittently generated at a frequency of between 0.2 and 10 Hz.

For some applications, probe system 600 comprises a light sensor 766, which is configured to be placed in the mouth or nose, or on an external surface of the face (e.g., the cheek) and to detect the light generated by light-emitting element 760. Light sensor 766 provides a signal to analysis processor 726 of position assessment unit 612, which analyzes the signal to ascertain whether the light-emitting element is adjacent to bone or soft tissue, i.e., whether the element is within the bony canal or in surrounding soft tissue. For example, the analysis may include comparing the measured intensity with a threshold value. Output unit 728 is configured to generate an output indicative of the result of the analysis. For example, the output may include an audible tone and/or a visible signal, and/or an electrical signal communicated to a separate monitoring device. Alternatively, the position assessment unit is configured to generate an output indicative of a value of the intensity of the sensed light, and the healthcare worker performing the implantation procedure ascertains whether the light-emitting element and shaft are within the bony canal responsively to the outputted value. For example, output unit 728 may include a display that displays a numerical value of the light intensity, and/or an audio generator that generates a tone having a frequency indicative of a magnitude of the light intensity.

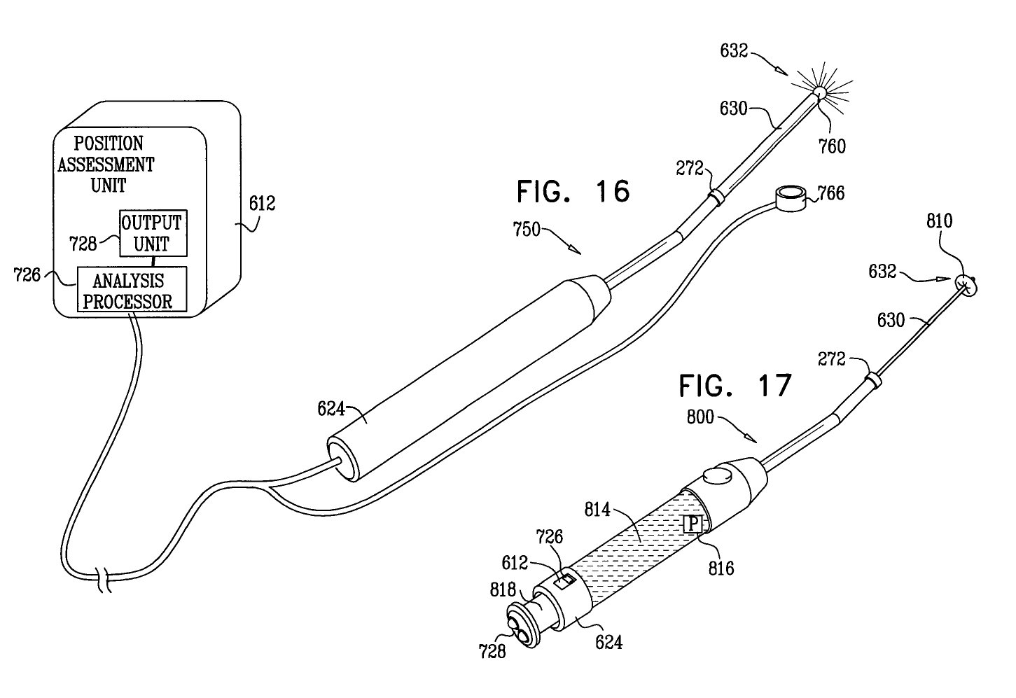

Reference is made to FIG. 17, which is a schematic illustration a balloon probe 800, in accordance with an embodiment of the present invention. In this embodiment, position assessment element 632 comprises a balloon 810 fixed to shaft 630, typically within 10 mm of a distal end thereof. In the figure, the balloon is shown having a short length along the shaft of between 1 and 2 mm; for other applications, the balloon is elongated, i.e., has a length along the shaft of between 2 and 5 mm, between 5 and 10 mm, or greater than 10 mm. Probe system 600 comprises a pressure-regulated fluid source 814, which is in fluid communication with the balloon via a channel along shaft 630 (either within the shaft or alongside the shaft). After the shaft is inserted into the body canal, fluid source 814 inflates the balloon with a volume of the fluid, while measuring the applied pressure. Position assessment unit 612 analyzes (a) the volume and/or rate of change of the volume and (b) the measured pressure and the provided fluid to ascertain whether the shaft is properly positioned in the body canal, as described hereinbelow.

For some applications, fluid source 814 is contained within handle 624, as shown in FIG. 17. Alternatively, the fluid source is provided in a separate housing that is coupled to the probe (configuration not shown). Fluid source 814 comprises a pressure sensor 816 for measuring the pressure of the fluid in balloon 810. Fluid source 814 also comprises a source of pressure, which may comprise a manual plunger 818 (as shown), or an automated pump (not shown). The fluid may comprise a liquid or a gas. Balloon 810 may be similar to a PTCA balloon, as is known in angioplasty art.

After the shaft and balloon are introduced into the bony canal, the source of pressure is activated to apply pressure to the fluid in fluid source 814, which inflates the balloon via the channel. If the balloon is properly positioned in the bony canal, the ratio of pressure to volume is relatively high. On the other hand, if the probe penetrates the wall of the canal into soft tissue surrounding the canal, the ratio of pressure to volume is relatively low.

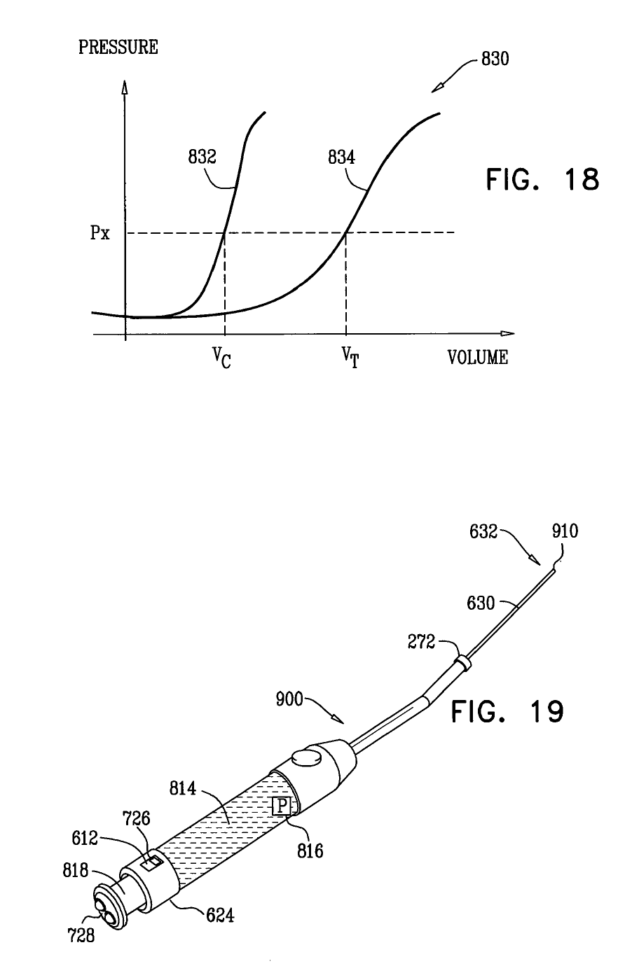

Reference is made to FIG. 18, which is a graph 830 showing two pressure-volume curves 832 and 834, in accordance with an embodiment of the present invention. In accordance with one technique for assessing whether the balloon is within the bony canal, pressure is applied to the fluid in fluid source 814 until pressure sensor 816 senses that the pressure in the fluid source reaches a predetermined fixed value Px. If the balloon is properly positioned in the bony canal, a first volume V_{C} of fluid has been provided by the fluid source to the balloon, as indicated on curve 832. On the other hand, if the shaft penetrates to wall of the canal into soft tissue surrounding the canal, such that the balloon is positioned in the soft tissue, a second volume V_{T} of fluid has been provided by the fluid source to the balloon, as indicated on curve 834. The second volume is greater than the first volume, because the resistance to inflation of the balloon within the bony canal is greater than the resistance provided by the soft tissue surrounding the canal. Thus, the ratio of pressure to volume is greater if the balloon is properly positioned within the canal than if improperly positioned in soft tissue surrounding the canal.

Alternatively, pressure is applied to the fluid in fluid source 814 until a predetermined fixed volume of fluid is pumped into the balloon. Pressure sensor 816 measures the resulting pressure. A relatively high pressure indicates that the balloon is properly positioned with the bony canal, while a relatively low pressure indicates the balloon is positioned in soft tissue surrounding the canal. The amount of time until a high pressure is achieved may also be used as an indication of whether the balloon is within the canal.

Other techniques for assessing the pressure/volume ratio will be evident to those skilled in the art, and are within the scope of the present invention. For some applications, balloon probe 800 does not comprise pressure sensor 816, and the pressure is instead estimated by the healthcare worker as he or she manually applies pressure to the fluid source.

Analysis processor 726 of position assessment unit 612 analyzes the ratio of pressure to volume, as described above, to ascertain whether the balloon is within the bony canal. For example, the analysis may include comparing the measured ratio with a threshold value. Output unit 728 is configured to generate an output indicative of the result of the analysis. For example, the output may include an audible tone and/or a visible signal, and/or an electrical signal communicated to a separate monitoring device. Alternatively, the position assessment unit is configured to generate an output indicative of a value of the ratio, and the healthcare worker performing the implantation procedure ascertains whether the balloon and shaft are within the bony canal responsively to the outputted value. For example, output unit 728 may include a display that displays a numerical value of the ratio, and/or an audio generator that generates a tone having a frequency indicative of a magnitude of the ratio.

Reference is made to FIG. 19, which is a schematic illustration of a fluid injection probe 900, in accordance with an embodiment of the present invention. Fluid injection probe 900 comprises the elements included in balloon probe 800, described hereinabove with reference to FIG. 17, with the exception of balloon 810. In this embodiment, position assessment element 632 comprises an opening 910, typically within 10 mm of a distal end of shaft 630, which opening is in fluid communication with pressure-regulated fluid source 814, via a channel along shaft 630 (either within the shaft or alongside the shaft). After the shaft is inserted into the body canal, fluid source 814 injects a volume of fluid through opening 910, such as saline solution, while measuring the applied pressure. Position assessment unit 612 analyzes (a) the volume and/or rate of change of the volume and (b) the measured pressure of fluid provided to ascertain whether the shaft is properly positioned in the body canal, using the analysis and output techniques described hereinabove with reference to FIGS. 17 and 18. The ratio of pressure to volume is greater if the fluid is injected into the body canal than if the fluid is injected into soft tissue. For some applications, fluid injection probe 900 does not comprise pressure sensor 816, and the pressure is instead estimated by the healthcare worker as he or she manually applies pressure to the fluid source.

In an embodiment of the present invention, system 600 comprises circuitry described in one or more of the patent applications incorporated herein by reference hereinbelow.

For some applications, the position assessment techniques described hereinabove with reference to FIGS. 13A-19 are used in combination with the positioning techniques described hereinabove with reference to FIGS. 9-12 and 7.

For some applications, instead of or in addition to being applied to SPG 210, electrodes are applied to another site of the subject, such as:
a nerve of the pterygoid canal (also called a vidian nerve), such as a greater superficial petrosal nerve (a preganglionic parasympathetic nerve) or a lesser deep petrosal nerve (a postganglionic sympathetic nerve);
a greater palatine nerve;
a lesser palatine nerve;
a sphenopalatine nerve;
a communicating branch between the maxillary nerve and the sphenopalatine ganglion;
an otic ganglion;
an afferent fiber going into the otic ganglion;
an efferent fiber going out of the otic ganglion; or
an infraorbital nerve.

For some applications, a neural stimulator (such as stimulator 422) is implanted using techniques described in US Patent Application Publication 2006/0195169 to Gross et al., which is assigned to the assignee of the present application and is incorporated herein by reference.

In an embodiment of the present invention, during placement of electrodes at SPG 210 or another site, at least one physiological indicator of cerebral blood flow (CBF) is observed or measured concurrently with or after placement. For some applications, optimization of placement of the electrodes onto the appropriate neural structure is performed by activating the stimulator, and generally simultaneously monitoring CBF while manipulating the electrodes, and/or adjusting at least one parameter of the applied stimulation, so as to increase or decrease CBF, as appropriate. Alternatively or additionally, this technique is used to verify the placement of the electrodes after implantation, and/or to select which combination of electrodes to use, such as by using the feedback algorithm described hereinabove. Alternatively or additionally, a similar optimization process is performed, either during or after placement of the electrodes, to determine parameters of the applied current so as to achieve a desired effect, e.g., on CBF or BBB permeability, as indicated by CBF.

Physiological indicators of CBF include, but are not limited to, the following:
blood flow in the common carotid artery, as measured by Doppler ultrasonography;
a measure of vasodilation of blood vessels of the eye, determined by unaided visual inspection or by using an instrument, e.g., an instrument comprising machine vision functionality;
transcranial Doppler ultrasonography measurements;
a measure of forehead perfusion, measured, for example, using laser Doppler perfusion imaging (LDI) and/or using a temperature sensor; and/or
near infrared spectroscopy (NIRS) measurements.

Other appropriate measurements indicative of CBF for use with these embodiments of the present invention will be apparent to those skilled in the art, having read the disclosure of the present patent application.

For some applications, one or more of the devices described in US Patent Application Publication 2006/0287677 with reference to FIGS. 19-22 thereof are used for assessing a physiological indicator of CBF.

In an embodiment of the present invention, during placement of electrodes at SPG 210 or another site, penetration of a systemically administered dye into an eye of the subject is observed or measured concurrently with or after placement, as an indication of a level of increased permeability of the BBB. For example, the dye may include fluorescein dye. For some applications, optimization of placement of the electrodes onto the appropriate neural structure is performed by activating the stimulator, and generally simultaneously monitoring the penetration of the dye while manipulating the electrodes, and/or adjusting at least one parameter of the applied stimulation, so as to increase or decrease permeability of the BBB, as appropriate. Alternatively or additionally, this technique is used to verify the placement of the electrodes after implantation, and/or to select which combination of electrodes to use, such as by using the feedback algorithm described hereinabove. Alternatively or additionally, a similar optimization process is performed, either during or after placement of the electrodes, to determine parameters of the applied current so as to achieve a desired effect, e.g., on CBF or BBB permeability, as indicated by BBB permeability.

In an embodiment of the present invention, one or more of the above-described CBF-based assessment techniques are used by a healthcare worker after implantation to assess (a) whether the electrodes retain appropriate placement and contact with the SPG or other site, and/or (b) whether parameters of the applied current (e.g., magnitude, frequency, duration, scheduling) continue to achieve the desired effect, e.g., on CBF or BBB permeability. For example, such an assessment may be performed periodically during post-implantation follow-up care.

It is to be appreciated that whereas some embodiments of the present invention are described with respect to implanting the electrical stimulator, for some applications the stimulator is temporarily inserted into the subject, and techniques described herein are used to optimize the temporary placement of the stimulator.

It is also to be appreciate that while some embodiments of the invention are generally described herein as enabling identification of the greater palatine canal, the techniques are also useful for identifying other bony canals, such as the incisive canal.

It is to be appreciated that while some embodiments of the invention are generally described herein with respect to electrical transmission of power and electrical modulation of tissue, other modes of energy transport may be used as well. Such energy includes, but is not limited to, direct or induced electromagnetic energy, radiofrequency (RF) transmission, mechanical vibration, ultrasonic transmission, optical power, and low power laser energy (via, for example, a fiber optic cable).

It is further to be appreciated that whereas some embodiments of the present invention are described with respect to application of electrical currents to tissue, this is to be understood in the context of the present patent application and in the claims as being substantially equivalent to applying an electrical field, e.g., by creating a voltage drop between two electrodes.

In some embodiments of the present invention, techniques described herein are practiced in combination with techniques described in one or more of the references cited in the Background of the Invention section hereinabove and/or in combination with techniques described in one or more of the patent applications cited hereinabove.

Techniques described in this application may be practiced in combination with methods and apparatus described in one or more of the following patent applications, which are assigned to the assignee of the present patent application and are incorporated herein by reference:
U.S. Provisional Patent Application 60/203,172, filed May 8, 2000, entitled, “Method and apparatus for stimulating the sphenopalatine ganglion to modify properties of the BBB and cerebral blood flow”
U.S. patent application Ser. No. 10/258,714, filed Oct. 25, 2002, entitled, “Method and apparatus for stimulating the sphenopalatine ganglion to modify properties of the BBB and cerebral blood flow,” or the above-referenced PCT Publication WO 01/85094
U.S. Provisional Patent Application 60/364,451, filed Mar. 15, 2002, entitled, “Applications of stimulating the sphenopalatine ganglion (SPG)”
U.S. Provisional Patent Application 60/368,657, filed Mar. 28, 2002, entitled, “SPG Stimulation”
U.S. Provisional Patent Application 60/376,048, filed Apr. 25, 2002, entitled, “Methods and apparatus for modifying properties of the BBB and cerebral circulation by using the neuroexcitatory and/or neuroinhibitory effects of odorants on nerves in the head”
U.S. Provisional Patent Application 60/388,931, filed Jun. 14, 2002, entitled “Methods and systems for management of Alzheimer's disease,” PCT Patent Application PCT/IL03/000508, filed Jun. 13, 2003, claiming priority therefrom, and a US patent application filed Dec. 14, 2004 in the national stage thereof
U.S. Provisional Patent Application 60/400,167, filed Jul. 31, 2002, entitled, “Delivering compounds to the brain by modifying properties of the BBB and cerebral circulation”
U.S. Provisional Patent Application 60/426,180, filed Nov. 14, 2002, entitled, “Surgical tools and techniques for sphenopalatine ganglion stimulation,” PCT Patent Application PCT/IL03/000966, filed Nov. 13, 2003, which claims priority therefrom, and a US patent application filed May 11, 2005 in the national stage thereof
U.S. Provisional Patent Application 60/426,182, filed Nov. 14, 2002, and corresponding PCT Patent Application PCT/IL03/000967, which claims priority therefrom, filed Nov. 13, 2003, entitled, “Stimulation circuitry and control of electronic medical device,” and a US patent application filed May 11, 2005 in the national stage thereof
U.S. patent application Ser. No. 10/294,310, filed Nov. 14, 2002, entitled, “SPG stimulation for treating eye pathologies,” which published as US Patent Application Publication 2003/0176898, and PCT Patent Application PCT/IL03/000965, filed Nov. 13, 2003, claiming priority therefrom
PCT Patent Application PCT/IL03/000631, filed Jul. 31, 2003, entitled, “Delivering compounds to the brain by modifying properties of the BBB and cerebral circulation,” which published as PCT Publication WO 04/010923, and U.S. patent application Ser. No. 10/522,615 in the national stage thereof
U.S. Pat. No. 6,853,858 to Shalev
U.S. patent application Ser. No. 10/783,113, filed Feb. 20, 2004, entitled, “Stimulation for acute conditions,” which published as US Patent Application Publication 2004/0220644
U.S. Provisional Patent Application 60/426,181, filed Nov. 14, 2002, entitled, “Stimulation for treating ear pathologies,” PCT Patent Application PCT/IL03/000963, filed Nov. 13, 2003, which claims priority therefrom, and which published as PCT Publication WO 04/045242, and U.S. patent application Ser. No. 10/535,025 in the national stage thereof
U.S. Provisional Patent Application 60/448,807, filed Feb. 20, 2003, entitled, “Stimulation for treating autoimmune-related disorders of the CNS”
U.S. Provisional Patent Application 60/461,232 to Gross et al., filed Apr. 8, 2003, entitled, “Treating abnormal conditions of the mind and body by modifying properties of the blood-brain barrier and cephalic blood flow”
PCT Patent Application PCT/IL03/00338 to Shalev, filed Apr. 25, 2003, entitled, “Methods and apparatus for modifying properties of the BBB and cerebral circulation by using the neuroexcitatory and/or neuroinhibitory effects of odorants on nerves in the head,” and U.S. patent application Ser. No. 10/512,780, filed Oct. 25, 2004 in the national stage thereof, which published as US Patent Application 2005/0266099
U.S. Provisional Patent Application 60/506,165, filed Sep. 26, 2003, entitled, “Diagnostic applications of stimulation”
U.S. patent application Ser. No. 10/678,730, filed Oct. 2, 2003, entitled, “Targeted release of nitric oxide in the brain circulation for opening the BBB,” which published as US Patent Application 2005/0074506, and PCT Patent Application PCT/IL04/000911, filed Oct. 3, 2004, claiming priority therefrom
PCT Patent Application PCT/IL04/000897, filed Sep. 26, 2004, entitled, “Stimulation for treating and diagnosing conditions,” which published as PCT Publication WO 05/030025
U.S. Provisional Patent Application 60/604,037, filed Aug. 23, 2004, entitled, “Concurrent bilateral SPG modulation”
PCT Patent Application PCT/IL05/000912, filed Aug. 23, 2005, entitled, “Concurrent bilateral SPG modulation,” which published as PCT Publication WO 06/021957
U.S. patent application Ser. No. 10/952,536, filed Sep. 27, 2004, entitled, “Stimulation for treating and diagnosing conditions,” which published as US Patent Application Publication 2005/0159790
U.S. patent application Ser. No. 11/349,020, filed Feb. 7, 2006, entitled, “SPG stimulation via the greater palatine canal”
U.S. patent application Ser. No. 11/465,381, filed Aug. 17, 2006, entitled, “Stimulation for treating brain events and other conditions”
U.S. patent application Ser. No. 11/668,305, filed Jan. 19, 2007, entitled, “Stimulation of the otic ganglion for treating medical conditions”
U.S. Provisional Application 61/195,556, filed Oct. 7, 2008, entitled, “Detection of electrode position for SPG stimulation by measurement of electrical effects”
U.S. patent application Ser. No. 12/197,614, filed Aug. 25, 2008, entitled, “SPG stimulation for enhancing neurogenesis and brain metabolism”
U.S. patent application Ser. No. 11/874,529, filed Oct. 18, 2007, entitled, “Long-term SPG stimulation therapy for prevention of vascular dementia”

It is noted that the figures depicting embodiments of the present invention are not necessarily drawn to scale, and, instead, may change certain dimensions in order to more clearly demonstrate some aspects of the invention.

It will be appreciated by persons skilled in the art that the present invention is not limited to what has been particularly shown and described hereinabove. Rather, the scope of the present invention includes both combinations and subcombinations of the various features described hereinabove, as well as variations and modifications thereof that are not in the prior art, which would occur to persons skilled in the art upon reading the foregoing description.

1. Apparatus comprising a surgical tool, which comprises:
a proximal shaft;
a distal rod, a proximal end thereof which is coupled to a distal end of the proximal shaft such that the distal rod articulates with the proximal shaft; and
a target sight, which comprises an aiming element, which is coupled to the distal rod and extends toward the proximal shaft, and which is indicative of an alignment of the distal rod with respect to the proximal shaft.

2. The apparatus according to claim 1, wherein the proximal shaft has a length of at least 50 mm, wherein the distal rod has a length of between 10 and 50 mm, and wherein a portion of the distal rod has a length of at least 5 mm and a greatest diameter of between 0.5 and 2 mm.

3. The apparatus according to claim 1, wherein the target sight further comprises an aiming ring, which is coupled to the proximal shaft, wherein the aiming element and the aiming ring are arranged such that the proximal shaft is generally centered with respect to the aiming ring when a central longitudinal axis of the distal element is parallel to a central longitudinal axis of the proximal shaft through a distal end of the proximal shaft.

4. The apparatus according to claim 1, wherein the proximal shaft is shaped so as to define a proximal shaft bend.

5. The apparatus according to claim 4, wherein the aiming element is shaped so as to define an aiming element bend having a same angle as the proximal shaft bend.

6. The apparatus according to claim 4, wherein the proximal shaft is shaped so as to define the proximal shaft bend at a location along the proximal shaft between 20 and 60 mm from a distal end of the distal rod, and wherein the proximal shaft bend has an angle of between 90 and 175 degrees.

7. The apparatus according to claim 1, wherein the distal rod and the proximal shaft are arranged to allow the distal rod to articulate with the proximal shaft in all directions.

8. The apparatus according to claim 7, wherein the proximal shaft is shaped so as to define a proximal shaft bend.

9. The apparatus according to claim 1, wherein the distal rod and the proximal shaft are arranged to allow the distal rod to articulate with the proximal shaft with greater than one degree of freedom.

10. A method comprising:
providing a surgical tool, which includes:
a proximal shaft,
a distal rod, a proximal end thereof which is coupled to a distal end of the proximal shaft such that the distal rod articulates with the proximal shaft, and
a target sight, which includes an aiming element, which is coupled to the distal rod and extends toward the proximal shaft, and which is indicative of an alignment of the distal rod with respect to the proximal shaft; and
preparing a greater palatine canal of a subject by:
advancing the surgical tool through at least a portion of the canal, and
ascertaining the alignment using the target sight.

11. The method according to claim 10, wherein preparing the canal further comprises, upon ascertaining that the distal rod is not properly aligned with respect to the proximal shaft, adjusting an orientation of the proximal shaft in order to properly align the distal rod with the shaft while ascertaining the alignment using the target sight.

12. The method according to claim 10, wherein the target sight further includes an aiming ring, which is coupled to the proximal shaft, wherein the aiming element and the aiming ring are arranged such that the proximal shaft is generally centered with respect to the aiming ring when a central longitudinal axis of the distal rod is parallel to a central longitudinal axis of the proximal shaft through a distal end of the proximal shaft, and wherein ascertaining the alignment comprises observing a position of the aiming element with respect to the aiming ring.

13. The method according to claim 10, wherein the distal rod and the proximal shaft are arranged to allow the distal rod to articulate with the proximal shaft in all directions.

14. The method according to claim 10, wherein the distal rod and the proximal shaft are arranged to allow the distal rod to articulate with the proximal shaft with greater than one degree of freedom.

15. Apparatus comprising a surgical tool, which comprises:
a proximal shaft; and
a distal rod, a proximal end thereof which is coupled to a distal end of the proximal shaft such that the distal rod articulates with the proximal shaft,
wherein the surgical tool is configured to allow the rod to articulate with the proximal shaft with exactly one degree of freedom, such that the rod, as it articulates with the one degree of freedom, defines a plane,
wherein a first portion of the distal rod facing in a first direction perpendicular to the plane is shaped so as to define an abrading surface, and wherein a second portion of the distal rod facing in a second direction opposite the first direction is not shaped so as to define an abrading surface.

16. The apparatus according to claim 15, wherein the proximal shaft is shaped so as to define a bend, which includes a radially outward bend portion and a radially inward bend portion, and wherein the first portion of the distal rod that is shaped so as to define the abrading surface faces generally in the same direction that the radially outward bend portion faces.

17. The apparatus according to claim 15, wherein the surgical tool further comprises a resisting element, which is arranged to resist articulation of the distal rod with the proximal shaft, and wherein the distal rod, the proximal shaft, and the resisting element are arranged to allow the rod to articulate with the proximal shaft with the exactly one degree of freedom.

18. The apparatus according to claim 17, wherein the resisting element is flat, such that it articulates with the exactly one degree of freedom.

19. The apparatus according to claim 17, wherein the resisting element couples the distal end of the proximal shaft to the proximal end of the distal rod.

20. The apparatus according to claim 17, wherein the resisting element comprises an elastic element.

21. A method comprising:
providing at least one surgical tool, which includes:
a proximal shaft, and
a distal rod, a proximal end thereof which is coupled to a distal end of the proximal shaft such that the distal rod articulates with the proximal shaft,
wherein the surgical tool is configured to allow the rod to articulate with the proximal shaft with exactly one degree of freedom such that the rod, as it articulates with the one degree of freedom, defines a plane,
wherein a first portion of the distal rod facing in a first direction perpendicular to the plane is shaped so as to define an abrading surface, and wherein a second portion of the distal rod facing in a second direction opposite the first direction is not shaped so as to define an abrading surface; and
preparing a greater palatine canal of a subject by:
advancing the surgical tool through at least a portion of the canal such that a distal tip of the distal rod applies a forward longitudinal force in order to open a passage through the canal, and
using the abrading surface to abrade a posterior wall of the canal.

22. The method according to claim 21, wherein the proximal shaft is shaped so as to define a bend, which includes a radially outward bend portion and a radially inward bend portion, and wherein the first portion of the distal rod that is shaped so as to define the abrading surface faces generally in the same direction that the radially outward bend portion faces, and wherein providing the at least one surgical tool comprises providing the at least one surgical tool shaped so as to define the bend.

23. The method according to claim 21, wherein the surgical tool further includes a resisting element, which is arranged to resist articulation of the distal rod with the proximal shaft, wherein the distal rod, the proximal shaft, and the resisting element are arranged to allow the rod to articulate with the proximal shaft with the exactly one degree of freedom, and wherein providing the at least one surgical tool comprises providing the at least one surgical tool including the resisting element.

24. The method according to claim 23, wherein the resisting element is elastic, and wherein providing the at least one surgical tool comprises providing the at least one surgical tool including the elastic element.

25.-44. (canceled)

45. Apparatus comprising a surgical kit, which comprises:
at least first and second surgical tools, each of which comprises:
a proximal shaft;
a distal rod, a proximal end thereof which is coupled to a distal end of the proximal shaft such that the distal rod articulates with the proximal shaft; and
a resisting element, which is arranged to resist articulation of the distal rod with the proximal shaft,
wherein the distal rod, the proximal shaft, and the resisting element of the first surgical are arranged to allow the rod to articulate with the proximal shaft with greater than one degree of freedom, and
wherein the distal rod, the proximal shaft, and the resisting element of the second surgical tool are arranged to allow the rod to articulate with the proximal shaft with exactly one degree of freedom, wherein the rod, as it articulates with the one degree of freedom, defines a plane, wherein a first portion of the distal rod of the second surgical tool facing in a first direction perpendicular to the plane is shaped so as to define an abrading surface, and wherein a second portion of the distal rod facing in a second direction opposite the first direction is not shaped so as to define an abrading surface.

46.-47. (canceled)

48. The apparatus according to claim 45, wherein the second surgical tool comprises at least the second surgical tool and a third surgical tool, and wherein a diameter of the first portion defining the abrading surface of the third surgical tool is greater than a diameter of the first portion defining the abrading surface of the second surgical tool.

49.-130. (canceled)
